SEQUENCE LISTING (23 sequences)

SEQ ID NO: 1 - protein, 657 aa, Homo sapiens
MEMETTEPEPDCVVQPPSPPDDFSCQMRLSEKITPLKTCFKKKDQKRLGTGTLRSLRPILNTLLESGSLDGVFRSRNQSTDENSLHEPMMKKAMEINSSCPPAENNMSVLIPDRTNVGDQIPEAHPSTEAPERVVPIQDHSFPSETLSGTVADSTPAHFQTDLLHPVSSDVPTSPDCLDKVIDYVPGIFQENSFTIQYILDTSDKLSTELFQDKSEEASLDLVFELVNQLQYHTHQENGIEICMDFLQGTCIYGRDCLKHHTVLPYHWQIKRTTTQKWQSVFNDSQEHLERFYCNPENDRMRMKYGGQEFWADLNAMNVYETTEFDQLRRLSTPPSSNVNSIYHTVWKFFCRDHFGWREYPESVIRLIEEANSRGLKEVRFMMWNNHYILHNSFFRREIKRRPLFRSCFILLPYLQTLGGVPTQAPPPLEATSSSQIICPDGVTSANFYPETWVYMHPSQDFIQVPVSAEDKSYRIIYNLFHKTVPEFKYRILQILRVQNQFLWEKYKRKKEYMNRKMFGRDRIINERHLFHGTSQDVVDGICKHNFDPRVCGKHATMFGQGSYFAKKASYSHNFSKKSSKGVHFMFLAKVLTGRYTMGSHGMRRPPPVNPGSVTSDLYDSCVDNFFEPQIFVIFNDDQSYPYFVIQYEEVSNTVSI

SEQ ID NO: 2 - protein, 1025 aa, Homo sapiens
MVAMAEAEAGVAVEVRGLPPAVPDELLTLYFENRRRSGGGPVLSWQRLGCGGVLTFREPADAERVLAQADHELHGAQLSLRPAPPRAPARLLLQGLPPGTTPQRLEQHVQALLRASGLPVQPCCALASPRPDRALVQLPKPLSEADVRVLEEQAQNLGLEGTLVSLARVPQARAVRVVGDGASVDLLLLELYLENERRSGGGPLEDLQRLPGPLGTVASFQQWQVAERVLQQEHRLQGSELSLVPHYDILEPEELAENTSGGDHPSTQGPRATKHALLRTGGLVTALQGAGTVTMGSGEEPGQSGASLRTGPMVQGRGIMTTGSGQEPGQSGTSLRTGPMGSLGQAEQVSSMPMGSLEHEGLVSLRPVGLQEQEGPMSLGPVGSAGPVETSKGLLGQEGLVEIAMDSPEQEGLVGPMEITMGSLEKAGPVSPGCVKLAGQEGLVEMVLLMEPGAMRFLQLYHEDLLAGLGDVALLPLEGPDMTGFRLCGAQASCQAAEEFLRSLLGSISCHVLCLEHPGSARFLLGPEGQHLLQGLEAQFQCVFGTERLATATLDTGLEEVDPTEALPVLPGNAHTLWTPDSTGGDQEDVSLEEVRELLATLEGLDLDGEDWLPRELEEEGPQEQPEEEVTPGHEEEEPVAPSTVAPRWLEEEAALQLALHRSLEPQGQVAEQEEAAALRQALTLSLLEQPPLEAEEPPDGGTDGKAQLVVHSAFEQDVEELDRALRAALEVHVQEETVGPWRRTLPAELRARLERCHGVSVALRGDCTILRGFGAHPARAARHLVALLAGPWDQSLAFPLAASGPTLAGQTLKGPWNNLERLAENTGEFQEVVRAFYDTLDAARSSIRVVRVERVSHPLLQQQYELYRERLLQRCERRPVEQVLYHGTTAPAVPDICAHGFNRSFCGRNATVYGKGVYFARRASLSVQDRYSPPNADGHKAVFVARVLTGDYGQGRRGLRAPPLRGPGHVLLRYDSAVDCICQPSIFVIFHDTQALPTHLITCEHVPRASPDDPSGLPGRSPDT

SEQ ID NO: 3 - protein, 701 aa, Homo sapiens
MAQAGVVGEVTQVLCAAGGALELPELRRRLRMGLSADALERLLRQRGRFVVAVRAGGAAAAPERVVLAASPLRLCRAHQGSKPGCVGLCAQLHLCRFMVYGACKFLRAGKNCRNSHSLTTEHNLSVLRTHGVDHLSYNELCQLLFQNDPWLLPEICQHYNKGDGPHGSCAFQKQCIKLHICQYFLQGECKFGTSCKRSHDFSNSENLEKLEKLGMSSDLVSRLPTIYRNAHDIKNKSSAPSRVPPLFVPQGTSERKDSSGSVSPNTLSQEEGDQICLYHIRKSCSFQDKCHRVHFHLPYRWQFLDRGKWEDLDNMELIEEAYCNPKIERILCSESASTFHSHCLNFNAMTYGATQARRLSTASSVTKPPHFILTTDWIWYWSDEFGSWQEYGRQGTVHPVTTVSSSDVEKAYLAYCTPGSDGQAATLKFQAGKHNYELDFKAFVQKNLVYGTTKKVCRRPKYVSPQDVTTMQTCNTKFPGPKSIPDYWDSSALPDPGFQKITLSSSSEEYQKVWNLFNRTLPFYFVQKIERVQNLALWEVYQWQKGQMQKQNGGKAVDERQLFHGTSAIFVDAICQQNFDWRVCGVHGTSYGKGSYFARDAAYSHHYSKSDTQTHTMFLARVLVGEFVRGNASFVRPPAKEGWSNAFYDSCVNSVSDPSIFVIFEKHQVYPEYVIQYTTSSKPSVTPSILLALGSLFSSRQ

SEQ ID NO: 4 - protein, 1801 aa, Homo sapiens
MAVPGSFPLLVEGSWGPDPPKNLNTKLQMYFQSPKRSGGGECEVRQDPRSPSRFLVFFYPEDVRQKVLERKNHELVWQGKGTFKLTVQLPATPDEIDHVFEEELLTKESKTKEDVKEPDVSEELDTKLPLDGGLDKMEDIPEECENISSLVAFENLKANVTDIMLILLVENISGLSNDDFQVEIIRDFDVAVVTFQKHIDTIRFVDDCTKHHSIKQLQLSPRLLEVTNTIRVENLPPGADDYSLKLFFENPYNGGGRVANVEYFPEESSALIEFFDRKVLDTIMATKLDFNKMPLSVFPYYASLGTALYGKEKPLIKLPAPFEESLDLPLWKFLQKKNHLIEEINDEMRRCHCELTWSQLSGKVTIRPAATLVNEGRPRIKTWQADTSTTLSSIRSKYKVNPIKVDPTMWDTIKNDVKDDRILIEFDTLKEMVILAGKSEDVQSIEVQVRELIESTTQKIKREEQSLKEKMIISPGRYFLLCHSSLLDHLLTECPEIEICYDRVTQHLCLKGPSADVYKAKCEIQEKVYTMAQKNIQVSPEIFQFLQQVNWKEFSKCLFIAQKILALYELEGTTVLLTSCSSEALLEAEKQMLSALNYKRIEVENKEVLHGKKWKGLTHNLLKKQNSSPNTVIINELTSETTAEVIITGCVKEVNETYKLLFNFVEQNMKIERLVEVKPSLVIDYLKTEKKLFWPKIKKVNVQVSFNPENKQKGILLTGSKTEVLKAVDIVKQVWDSVCVKSVHTDKPGAKQFFQDKARFYQSEIKRLFGCYIELQENEVMKEGGSPAGQKCFSRTVLAPGVVLIVQQGDLARLPVDVVVNASNEDLKHYGGLAAALSKAAGPELQADCDQIVKREGRLLPGNATISKAGKLPYHHVIHAVGPRWSGYEAPRCVYLLRRAVQLSLCLAEKYKYRSIAIPAISSGVFGFPLGRCVETIVSAIKENFQFKKDGHCLKEIYLVDVSEKTVEAFAEAVKTVFKATLPDTAAPPGLPPAAAGPGKTSWEKGSLVSPGGLQMLLVKEGVQNAKTDVVVNSVPLDLVLSRGPLSKSLLEKAGPELQEELDTVGQGVAVSMGTVLKTSSWNLDCRYVLHVVAPEWRNGSTSSLKIMEDIIRECMEITESLSLKSIAFPAIGTGNLGFPKNIFAELIISEVFKFSSKNQLKTLQEVHFLLHPSDHENIQAFSDEFARRANGNLVSDKIPKAKDTQGFYGTVSSPDSGVYEMKIGSIIFQVASGDITKEEADVIVNSTSNSFNLKAGVSKAILECAGQNVERECSQQAQQRKNDYIITGGGFLRCKNIIHVIGGNDVKSSVSSVLQECEKKNYSSICLPAIGTGNAKQHPDKVAEAIIDAIEDFVQKGSAQSVKKVKVVIFLPQVLDVFYANMKKREGTQLSSQQSVMSKLASFLGFSKQSPQKKNHLVLEKKTESATFRVCGENVTCVEYAISWLQDLIEKEQCPYTSEDECIKDFDEKEYQELNELQKKLNINISLDHKRPLIKVLGISRDVMQARDEIEAMIKRVRLAKEQESRADCISEFIEWQYNDNNTSHCFNKMTNLKLEDARREKKKTVDVKINHRHYTVNLNTYTATDTKGHSLSVQRLTKSKVDIPAHWSDMKQQNFCVVELLPSDPEYNTVASKFNQTCSHFRIEKIERIQNPDLWNSYQAKKKTMDAKNGQTMNEKQLFHGTDAGSVPHVNRNGFNRSYAGKNAVAYGKGTYFAVNANYSANDTYSRPDANGRKHVYYVRVLTGIYTHGNHSLIVPPSKNPQNPTDLYDTVTDNVHHPSLFVAFYDYQAYPEYLITFRK

SEQ ID NO: 5 - protein, 444 aa, Homo sapiens
MLQRIGLIFLHNIVVVSNCFYFQAFLDEFTNWSRINPNKARIPMAGDTQGVVGTVSKPCFTAYEMKIGAITFQVATGDIATEQVDVIVNSTARTFNRKSGVSRAILEGAGQAVESECAVLAAQPHRDFIITPGGCLKCKIIIHVPGGKDVRKTVTSVLEECEQRKYTSVSLPAIGTGNAGKNPITVADNIIDAIVDFSSQHSTPSLKTVKVVIFQPELLNIFYDSMKKRDLSASLNFQSTFSMTTCNLPEHWTDMNHQLFCMVQLEPGQSEYNTIKDKFTRTCSSYAIEKIERIQNAFLWQSYQVKKRQMDIKNDHKNNERLLFHGTDADSVPYVNQHGFNRSCAGKNAVSYGKGTYFAVDASYSAKDTYSKPDSNGRKHMYVVRVLTGVFTKGRAGLVTPPPKNPHNPTDLFDSVTNNTRSPKLFVVFFDNQAYPEYLITFTA

SEQ ID NO: 6 - protein, 323 aa, Homo sapiens
MQPSGWAAAREAAGRDMLAADLRCSLFASALQSYKRDSVLRPFPASYARGDCKDFEALLADASKLPNLKELLQSSGDNHKRAWDLVSWILSSKVLTIHSAGKAEFEKIQKLTGAPHTPVPAPDFLFEIEYFDPANAKFYETKGERDLIYAFHGSRLENFHSIIHNGLHCHLNKTSLFGEGTYLTSDLSLALIYSPHGHGWQHSLLGPILSCVAVCEVIDHPDVKCQTKKKDSKEIDRRRARIKHSEGGDIPPKYFVVTNNQLLRVKYLLVYSQKPPKSRASSQLSWFSSHWFTVMISLYLLLLLIVSVINSSAFQHFWNRAKR

SEQ ID NO: 7 - protein, 39 aa, synthetic construct
MHHHHHHSSGVDLGTENLYFQSNAGLNDIFEAQKIEWHE

SEQ ID NO: 8 - protein, 16 aa, synthetic construct
MAHHHHHHENLYFQSM

SEQ ID NO: 9 - protein, 24 aa, synthetic construct
MHHHHHHSSGVDLGTENLYFQSNA

SEQ ID NO: 10 - protein, 24 aa, synthetic construct
MAHHHHHHSSGVDLGTENLYFQSM

SEQ ID NO: 11 - protein, 171 aa, synthetic construct
MVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILA

SEQ ID NO: 12 - DNA, 516 bp, synthetic construct
  source 1..516: mol_type = other DNA
atggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaa

SEQ ID NO: 13 - protein, 1014 aa, Homo sapiens
MAESSDKLYRVEYAKSGRASCKKCSESIPKDSLRMAIMVQSPMFDGKVPHWYHFSCFWKVGHSIRHPDVEVDGFSELRWDDQQKVKKTAEAGGVTGKGQDGIGSKAEKTLGDFAAEYAKSNRSTCKGCMEKIEKGQVRLSKKMVDPEKPQLGMIDRWYHPGCFVKNREELGFRPEYSASQLKGFSLLATEDKEALKKQLPGVKSEGKRKGDEVDGVDEVAKKKSKKEKDKDSKLEKALKAQNDLIWNIKDELKKVCSTNDLKELLIFNKQQVPSGESAILDRVADGMVFGALLPCEECSGQLVFKSDAYYCTGDVTAWTKCMVKTQTPNRKEWVTPKEFREISYLKKLKVKKQDRIFPPETSASVAATPPPSTASAPAAVNSSASADKPLSNMKILTLGKLSRNKDEVKAMIEKLGGKLTGTANKASLCISTKKEVEKMNKKMEEVKEANIRVVSEDFLQDVSASTKSLQELFLAHILSPWGAEVKAEPVEVVAPRGKSGAALSKKSKGQVKEEGINKSEKRMKLTLKGGAAVDPDSGLEHSAHVLEKGGKVFSATLGLVDIVKGTNSYYKLQLLEDDKENRYWIFRSWGRVGTVIGSNKLEQMPSKEDAIEHFMKLYEEKTGNAWHSKNFTKYPKKFYPLEIDYGQDEEAVKKLTVNPGTKSKLPKPVQDLIKMIFDVESMKKAMVEYEIDLQKMPLGKLSKRQIQAAYSILSEVQQAVSQGSSDSQILDLSNRFYTLIPHDFGMKKPPLLNNADSVQAKVEMLDNLLDIEVAYSLLRGGSDDSSKDPIDVNYEKLKTDIKVVDRDSEEAEIIRKYVKNTHATTHNAYDLEVIDIFKIEREGECQRYKPFKQLHNRRLLWHGSRTTNFAGILSQGLRIAPPEAPVTGYMFGKGIYFADMVSKSANYCHTSQGDPIGLILLGEVALGNMYELKHASHISKLPKGKHSVKGLGKTTPDPSANISLDGVDVPLGTGISSGVNDTSLLYNEYIVYDIAQVNLKYLLKLKFNFKTSLW

SEQ ID NO: 14 - protein, 583 aa, Homo sapiens
MAARRRRSTGGGRARALNESKRVNNGNTAPEDSSPAKKTRRCQRQESKKMPVAGGKANKDRTEDKQDGMPGRSWASKRVSESVKALLLKGKAPVDPECTAKVGKAHVYCEGNDVYDVMLNQTNLQFNNNKYYLIQLLEDDAQRNFSVWMRWGRVGKMGQHSLVACSGNLNKAKEIFQKKFLDKTKNNWEDREKFEKVPGKYDMLQMDYATNTQDEEETKKEESLKSPLKPESQLDLRVQELIKLICNVQAMEEMMMEMKYNTKKAPLGKLTVAQIKAGYQSLKKIEDCIRAGQHGRALMEACNEFYTRIPHDFGLRTPPLIRTQKELSEKIQLLEALGDIEIAIKLVKTELQSPEHPLDQHYRNLHCALRPLDHESYEFKVISQYLQSTHAPTHSDYTMTLLDLFEVEKDGEKEAFREDLHNRMLLWHGSRMSNWVGILSHGLRIAPPEAPITGYMFGKGIYFADMSSKSANYCFASRLKNTGLLLLSEVALGQCNELLEANPKAEGLLQGKHSTKGLGKMAPSSAHFVTLNGSTVPLGPASDTGILNPDGYTLNYNEYIVYNPNQVRMRYLLKVQFNFLQLW

SEQ ID NO: 15 - protein, 540 aa, Homo sapiens
MSLLFLAMAPKPKPWVQTEGPEKKKGRQAGREEDPFRSTAEALKAIPAEKRIIRVDPTCPLSSNPGTQVYEDYNCTLNQTNIENNNNKFYIIQLLQDSNRFFTCWNRWGRVGEVGQSKINHFTRLEDAKKDFEKKFREKTKNNWAERDHFVSHPGKYTLIEVQAEDEAQEAVVKVDRGPVRTVTKRVQPCSLDPATQKLITNIFSKEMFKNTMALMDLDVKKMPLGKLSKQQIARGFEALEALEEALKGPTDGGQSLEELSSHFYTVIPHNFGHSQPPPINSPELLQAKKDMLLVLADIELAQALQAVSEQEKTVEEVPHPLDRDYQLLKCQLQLLDSGAPEYKVIQTYLEQTGSNHRCPTLQHIWKVNQEGEEDRFQAHSKLGNRKLLWHGTNMAVVAAILTSGLRIMPHSGGRVGKGIYFASENSKSAGYVIGMKCGAHHVGYMFLGEVALGREHHINTDNPSLKSPPPGFDSVIARGHTEPDPTQDTELELDGQQVVVPQGQPVPCPEFSSSTFSQSEYLIYQESQCRLRYLLEVHL

SEQ ID NO: 16 - protein, 1724 aa, Homo sapiens
MVMGIFANCIFCLKVKYLPQQQKKKLQTDIKENGGKFSFSLNPQCTHIILDNADVLSQYQLNSIQKNHVHIANPDFIWKSIREKRLLDVKNYDPYKPLDITPPPDQKASSSEVKTEGLCPDSATEEEDTVELTEFGMQNVEIPHLPQDFEVAKYNTLEKVGMEGGQEAVVVELQCSRDSRDCPFLISSHFLLDDGMETRRQFAIKKTSEDASEYFENYIEELKKQGFLLREHFTPEATQLASEQLQALLLEEVMNSSTLSQEVSDLVEMIWAEALGHLEHMLLKPVNRISLNDVSKAEGILLLVKAALKNGETAEQLQKMMTEFYRLIPHKGTMPKEVNLGLLAKKADLCQLIRDMVNVCETNLSKPNPPSLAKYRALRCKIEHVEQNTEEFLRVRKEVLQNHHSKSPVDVLQIFRVGRVNETTEFLSKLGNVRPLLHGSPVQNIVGILCRGLLLPKVVEDRGVQRTDVGNLGSGIYFSDSLSTSIKYSHPGETDGTRLLLICDVALGKCMDLHEKDFSLTEAPPGYDSVHGVSQTASVTTDFEDDEFVVYKTNQVKMKYIIKFSMPGDQIKDFHPSDHTELEEYRPEFSNFSKVEDYQLPDAKTSSSTKAGLQDASGNLVPLEDVHIKGRIIDTVAQVIVFQTYTNKSHVPIEAKYIFPLDDKAAVCGFEAFINGKHIVGEIKEKEEAQQEYLEAVTQGHGAYLMSQDAPDVFTVSVGNLPPKAKVLIKITYITELSILGTVGVFFMPATVAPWQQDKALNENLQDTVEKICIKEIGTKQSFSLTMSIEMPYVIEFIFSDTHELKQKRTDCKAVISTMEGSSLDSSGFSLHIGLSAAYLPRMWVEKHPEKESEACMLVFQPDLDVDLPDLASESEVIICLDCSSSMEGVTFLQAKQIALHALSLVGEKQKVNIIQFGTGYKELFSYPKHITSNTMAAEFIMSATPTMGNTDFWKTLRYLSLLYPARGSRNILLVSDGHLQDESLTLQLVKRSRPHTRLFACGIGSTANRHVLRILSQCGAGVFEYFNAKSKHSWRKQIEDQMTRLCSPSCHSVSVKWQQLNPDVPEALQAPAQVPSLFLNDRLLVYGFIPHCTQATLCALIQEKEFRTMVSTTELQKTTGTMIHKLAARALIRDYEDGILHENETSHEMKKQTLKSLIIKLSKENSLITQFTSFVAVEKRDENESPFPDIPKVSELIAKEDVDFLPYMSWQGEPQEAVRNQSLLASSEWPELRLSKRKHRKIPFSKRKMELSQPEVSEDFEEDGLGVLPAFTSNLERGGVEKLLDLSWTESCKPTATEPLFKKVSPWETSTSSFFPILAPAVGSYLPPTARAHSPASLSFASYRQVASFGSAAPPRQFDASQFSQGPVPGTCADWIPQSASCPTGPPQNPPSSPYCGIVFSGSSLSSAQSAPLQHPGGFTTRPSAGTFPELDSPQLHFSLPTDPDPIRGFGSYHPSASSPFHFQPSAASLTANLRLPMASALPEALCSQSRTTPVDLCLLEESVGSLEGSRCPVFAFQSSDTESDELSEVLQDSCFLQIKCDTKDDSILCFLEVKEEDEIVCIQHWQDAVPWTELLSLQTEDGFWKLTPELGLILNLNTNGLHSFLKQKGIQSLGVKGRECLLDLIATMLVLQFIRTRLEKEGIVFKSLMKMDDASISRNIPWAFEAIKQASEWVRRTEGQYPSICPRLELGNDWDSATKQLLGLQPISTVSPLHRVLHYSQG

SEQ ID NO: 17 - protein, 1327 aa, Homo sapiens
MAASRRSQHHHHHHQQQLQPAPGASAPPPPPPPPLSPGLAPGTTPASPTASGLAPFASPRHGLALPEGDGSRDPPDRPRSPDPVDGTSCCSTTSTICTVAAAPVVPAVSTSSAAGVAPNPAGSGSNNSPSSSSSPTSSSSSSPSSPGSSLAESPEAAGVSSTAPLGPGAAGPGTGVPAVSGALRELLEACRNGDVSRVKRLVDAANVNAKDMAGRKSSPLHFAAGFGRKDVVEHLLQMGANVHARDDGGLIPLHNACSFGHAEVVSLLLCQGADPNARDNWNYTPLHEAAIKGKIDVCIVLLQHGADPNIRNTDGKSALDLADPSAKAVLTGEYKKDELLEAARSGNEEKLMALLTPLNVNCHASDGRKSTPLHLAAGYNRVRIVQLLLQHGADVHAKDKGGLVPLHNACSYGHYEVTELLLKHGACVNAMDLWQFTPLHEAASKNRVEVCSLLLSHGADPTLVNCHGKSAVDMAPTPELRERLTYEFKGHSLLQAAREADLAKVKKTLALEIINFKQPQSHETALHCAVASLHPKRKQVTELLLRKGANVNEKNKDFMTPLHVAAERAHNDVMEVLHKHGAKMNALDTLGQTALHRAALAGHLQTCRLLLSYGSDPSIISLQGFTAAQMGNEAVQQILSESTPIRTSDVDYRLLEASKAGDLETVKQLCSSQNVNCRDLEGRHSTPLHFAAGYNRVSVVEYLLHHGADVHAKDKGGLVPLHNACSYGHYEVAELLVRHGASVNVADLWKFTPLHEAAAKGKYEICKLLLKHGADPTKKNRDGNTPLDLVKEGDTDIQDLLRGDAALLDAAKKGCLARVQKLCTPENINCRDTQGRNSTPLHLAAGYNNLEVAEYLLEHGADVNAQDKGGLIPLHNAASYGHVDIAALLIKYNTCVNATDKWAFTPLHEAAQKGRTQLCALLLAHGADPTMKNQEGQTPLDLATADDIRALLIDAMPPEALPTCFKPQATVVSASLISPASTPSCLSAASSIDNLTGPLAELAVGGASNAGDGAAGTERKEGEVAGLDMNISQFLKSLGLEHLRDIFETEQITLDVLADMGHEELKEIGINAYGHRHKLIKGVERLLGGQQGTNPYLTFHCVNQGTILLDLAPEDKEYQSVEEEMQSTIREHRDGGNAGGIFNRYNVIRIQKVVNKKLRERFCHRQKEVSEENHNHHNERMLFHGSPFINAIIHKGFDERHAYIGGMFGAGIYFAENSSKSNQYVYGIGGGTGCPTHKDRSCYICHRQMLFCRVTLGKSFLQFSTMKMAHAPPGHHSVIGRPSVNGLAYAEYVIYRGEQAYPEYLITYQIMKPEAPSQTATAAEQKT

SEQ ID NO: 18 - protein, 1166 aa, Homo sapiens
MSGRRCAGGGAACASAAAEAVEPAARELFEACRNGDVERVKRLVTPEKVNSRDTAGRKSTPLHFAAGFGRKDVVEYLLQNGANVQARDDGGLIPLHNACSFGHAEVVNLLLRHGADPNARDNWNYTPLHEAAIKGKIDVCIVLLQHGAEPTIRNTDGRTALDLADPSAKAVLTGEYKKDELLESARSGNEEKMMALLTPLNVNCHASDGRKSTPLHLAAGYNRVKIVQLLLQHGADVHAKDKGDLVPLHNACSYGHYEVTELLVKHGACVNAMDLWQFTPLHEAASKNRVEVCSLLLSYGADPTLLNCHNKSAIDLAPTPQLKERLAYEFKGHSLLQAAREADVTRIKKHLSLEMVNFKHPQTHETALHCAAASPYPKRKQICELLLRKGANINEKTKEFLTPLHVASEKAHNDVVEVVVKHEAKVNALDNLGQTSLHRAAYCGHLQTCRLLLSYGCDPNIISLQGFTALQMGNENVQQLLQEGISLGNSEADRQLLEAAKAGDVETVKKLCTVQSVNCRDIEGRQSTPLHFAAGYNRVSVVEYLLQHGADVHAKDKGGLVPLHNACSYGHYEVAELLVKHGAVVNVADLWKFTPLHEAAAKGKYEICKLLLQHGADPTKKNRDGNTPLDLVKDGDTDIQDLLRGDAALLDAAKKGCLARVKKLSSPDNVNCRDTQGRHSTPLHLAAGYNNLEVAEYLLQHGADVNAQDKGGLIPLHNAASYGHVDVAALLIKYNACVNATDKWAFTPLHEAAQKGRTQLCALLLAHGADPTLKNQEGQTPLDLVSADDVSALLTAAMPPSALPSCYKPQVLNGVRSPGATADALSSGPSSPSSLSAASSLDNLSGSFSELSSVVSSSGTEGASSLEKKEVPGVDFSITQFVRNLGLEHLMDIFEREQITLDVLVEMGHKELKEIGINAYGHRHKLIKGVERLISGQQGLNPYLTLNTSGSGTILIDLSPDDKEFQSVEEEMQSTVREHRDGGHAGGIFNRYNILKIQKVCNKKLWERYTHRRKEVSEENHNHANERMLFHGSPFVNAIIHKGFDERHAYIGGMFGAGIYFAENSSKSNQYVYGIGGGTGCPVHKDRSCYICHRQLLFCRVTLGKSFLQFSAMKMAHSPPGHHSVTGRPSVNGLALAEYVIYRGEQAYPEYLITYQIMRPEGMVDG

SEQ ID NO: 19 - protein, 630 aa, Homo sapiens
MDIKGQFWNDDDSEGDNESEEFLYGVQGSCAADLYRHPQLDADIEAVKEIYSENSVSIREYGTIDDVDIDLHINISFLDEEVSTAWKVLRTEPIVLRLRFSLSQYLDGPEPSIEVFQPSNKEGFGLGLQLKKILGMFTSQQWKHLSNDFLKTQQEKRHSWFKASGTIKKFRAGLSIFSPIPKSPSFPIIQDSMLKGKLGVPELRVGRLMNRSISCTMKNPKVEVFGYPPSPQAGLLCPQHVGLPPPARTSPLVSGHCKNIPTLEYGFLVQIMKYAEQRIPTLNEYCVVCDEQHVFQNGSMLKPAVCTRELCVFSFYTLGVMSGAAEEVATGAEVVDLLVAMCRAALESPRKSIIFEPYPSVVDPTDPKTLAFNPKKKNYERLQKALDSVMSIREMTQGSYLEIKKQMDKLDPLAHPLLQWIISSNRSHIVKLPLSRLKFMHTSHQFLLLSSPPAKEARFRTAKKLYGSTFAFHGSHIENWHSILRNGLVNASYTKLQLHGAAYGKGIYLSPISSISFGYSGMGKGQHRMPSKDELVQRYNRMNTIPQTRSIQSRFLQSRNLNCIALCEVITSKDLQKHGNIWVCPVSDHVCTRFFFVYEDGQVGDANINTQDPKIQKEIMRVIGTQVYTN

SEQ ID NO: 20 - protein, 494 aa, Homo sapiens
MQTFVTQQWKQSKEKSNCLHNKKLSEKKVKSPLHLFSTLRRSPSYPPPGCGKSKSKLKSEQDGISKTHKLLRRTCSSTVKTDDVCVTKSHRTFGRSLSSDPRAEQAMTAIKSHKLLNRPCPAAVKSEECLTLKSHRLLTRSCSGDPRCEHNTNLKPHKLLSRSYSSNLRMEELYGLKNHKLLSKSYSSAPKSSKTELFKEPNAEGRRLSLTSGLIGILTPSSSSSSQLAPNGAKCIPVRDRGFLVQTIEFAEQRIPVLNEYCVVCDEPHVFQNGPMLRPTVCERELCVFAFQTLGVMNEAADEIATGAQKKNYDRVMKALDSITSIREMTQAPYLEIKKQMDKQDPLAHPLLQWVISSNRSHIVKLPVNRQLKFMHTPHQFLLLSSPPAKESNFRAAKKLFGSTFAFHGSHIENWHSILRNGLVVASNTRLQLHGAMYGSGIYLSPMSSISFGYSGMNKKQKVSAKDEPASSSKSSNTSQSQKKRTAIPIPAKP

SEQ ID NO: 21 - protein, 819 aa, Homo sapiens
MDFSMVAGAAAYNEKSETGALGENYSWQIPINHNDFKILKNNERQLCEVLQNKFGCISTLVSPVQEGNSKSLQVFRKMLTPRIELSVWKDDLTTHAVDAVVNAANEDLLHGGGLALALVKAGGFEIQEESKQFVARYGKVSAGEIAVTGAGRLPCKQIIHAVGPRWMEWDKQGCTGKLQRAIVSILNYVIYKNTHIKTVAIPALSSGIFQFPLNLCTKTIVETIRVSLQGKPMMSNLKEIHLVSNEDPTVAAFKAASEFILGKSELGQETTPSFNAMVVNNLTLQIVQGHIEWQTADVIVNSVNPHDITVGPVAKSILQQAGVEMKSEFLATKAKQFQRSQLVLVTKGFNLFCKYIYHVLWHSEFPKPQILKHAMKECLEKCIEQNITSISFPALGTGNMEIKKETAAEILFDEVLTFAKDHVKHQLTVKFVIFPTDLEIYKAFSSEMAKRSKMLSLNNYSVPQSTREEKRENGLEARSPAINLMGFNVEEMYEAHAWIQRILSLQNHHIIENNHILYLGRKEHDILSQLQKTSSVSITEIISPGRTELEIEGARADLIEVVMNIEDMLCKVQEEMARKKERGLWRSLGQWTIQQQKTQDEMKENIIFLKCPVPPTQELLDQKKQFEKCGLQVLKVEKIDNEVLMAAFQRKKKMMEEKLHRQPVSHRLFQQVPYQFCNVVCRVGFQRMYSTPCDPKYGAGIYFTKNLKNLAEKAKKISAADKLIYVFEAEVLTGFFCQGHPLNIVPPPLSPGAIDGHDSVVDNVSSPETFVIFSGMQAIPQYLWTCTQEYVQSQDYSSGPMRPFAQHPWRGFASGSPVD

SEQ ID NO: 22 - protein, 338 aa, Homo sapiens
MWEANPEMFHKAEELFSKTTNNEVDDMDTSDTQWGWFYLAECGKWHMFQPDTNSQCSVSSEDIEKSFKTNPCGSISFTTSKFSYKIDFAEMKQMNLTTGKQRLIKRAPFSISAFSYICENEAIPMPPHWENVNTQVPYQLIPLHNQTHEYNEVANLFGKTMDRNRIKRIQRIQNLDLWEFFCRKKAQLKKKRGVPQINEQMLFHGTSSEFVEAICIHNFDWRINGIHGAVFGKGTYFARDAAYSSRFCKDDIKHGNTFQIHGVSLQQRHLFRTYKSMFLARVLIGDYINGDSKYMRPPSKDGSYVNLYDSCVDDTWNPKIFVVFDANQIYPEYLIDFH

SEQ ID NO: 23 - protein, 902 aa, Homo sapiens
MADPEVCCFITKILCAHGGRMALDALLQEIALSEPQLCEVLQVAGPDRFVVLETGGEAGITRSVVATTRARVCRRKYCQRPCDNLHLCKLNLLGRCNYSQSERNLCKYSHEVLSEENFKVLKNHELSGLNKEELAVLLLQSDPFFMPEICKSYKGEGRQQICNQQPPCSRLHICDHFTRGNCRFPNCLRSHNLMDRKVLAIMREHGLNPDVVQNIQDICNSKHMQKNPPGPRAPSSHRRNMAYRARSKSRDRFFQGSQEFLASASASAERSCTPSPDQISHRASLEDAPVDDLTRKFTYLGSQDRARPPSGSSKATDLGGTSQAGTSQRFLENGSQEDLLHGNPGSTYLASNSTSAPNWKSLTSWTNDQGARRKTVFSPTLPAARSSLGSLQTPEAVTTRKGTGLLSSDYRIINGKSGTQDIQPGPLFNNNADGVATDITSTRSLNYKSTSSGHREISSPRIQDAGPASRDVQATGRIADDADPRVALVNDSLSDVTSTTSSRVDDHDSEEICLDHLCKGCPLNGSCSKVHFHLPYRWQMLIGKTWTDFEHMETIEKGYCNPGIHLCSVGSYTINFRVMSCDSFPIRRLSTPSSVTKPANSVFTTKWIWYWKNESGTWIQYGEEKDKRKNSNVDSSYLESLYQSCPRGVVPFQAGSRNYELSFQGMIQTNIASKTQKDVIRRPTFVPQWYVQQMKRGPDHQPAKTSSVSLTATFRPQEDFCFLSSKKYKLSEIHHLHPEYVRVSEHFKASMKNFKIEKIKKIENSELLDKFTWKKSQMKEEGKLLFYATSRAYVESICSNNFDSFLHETHENKYGKGIYFAKDAIYSHKNCPYDAKNVVMFVAQVLVGKFTEGNITYTSPPPQFDSCVDTRSNPSVFVIFQKDQVYPQYVIEYTEDKACVIS

(19) United States
(12) Patent Application Publication
(10) Pub. No.: US 2025/0020654 A1
(43) Pub. Date: Jan. 16, 2025

(54) SCREENING METHODS FOR PARP MODULATORS

(71) Applicant: Ribon Therapeutics, Inc., Cambridge, MA (US)

(72) Inventors: Timothy J.N. Wigle, Cambridge, MA (US); Danielle J. Blackwell, Medford, MA (US); Kevin Wayne Kuntz, Woburn, MA (US); Melissa Marie Vasbinder, Newton, MA (US); Laurie B. Schenkel, Somerville, MA (US); Kerren Kalai Swinger, Lexington, MA (US)

(21) Appl. No.: 18/745,589

(22) Filed: Jun. 17, 2024

Related U.S. Application Data
(63) Continuation of application No. 17/697,322, filed on Mar. 17, 2022, now abandoned.
Application No. 17/697,322 is a division of application No. 16/397,410, filed on Apr. 29, 2019, now Pat. No. 11,293,927.
(60) Provisional application No. 62/664,595, filed on Apr. 30, 2018.

(51) Int. Cl. G01N 33/58; C07D 491/22; C07D 493/10; C07D 495/04; C07F 5/02; G01N 33/573

(52) U.S. Cl. CPC G01N 33/582; C07D 491/22; C07D 493/10; C07D 495/04; C07F 5/027; G01N 33/573; G01N 2333/91142

(57) ABSTRACT

The present disclosure is related to methods of identifying Poly(ADP-ribose) polymerases (PARP) inhibitors, and the methods of using PARP probes.

### CLAIM OF PRIORITY

This application is a continuation application of U.S. patent application Ser. No. 17/697,322, filed on Mar. 17, 2022, which is a divisional application of U.S. patent application Ser. No. 16/397,410, filed on Apr. 29, 2019, which claims priority to U.S. Provisional Patent Application Ser. No. 62/664,595, filed on Apr. 30, 2018, the entire contents of which are hereby incorporated by reference.

### SEQUENCE LISTING

This application contains a Sequence Listing that has been submitted electronically as an XML file named “42921-0007003_SL_ST26.XML.” The XML file, created on Jun. 17, 2024, is 35,907 bytes in size. The material in the XML file is hereby incorporated by reference in its entirety.

### TECHNICAL FIELD

The present disclosure is related to methods of identifying Poly(ADP-ribose) polymerase (PARP) inhibitors.

### BACKGROUND

Poly(ADP-ribose) polymerases (PARPs) are a family of 17 enzymes that can transfer ADP-ribose from nicotinamide adenine dinucleotide (NAD⁺) to protein and nucleic acid substrates¹-2. The PARP enzymes family is comprised of two subfamilies, monoPARPs and polyPARPs. MonoPARP enzymes catalyze the transfer of a single ADP-ribose group to a target amino acid, while polyPARP enzymes catalyze the transfer of multiple ADP-ribose groups to form polymers. While approved drugs that target the polyPARPs exist, there are no potent and selective inhibitors of the monoPARPs. MonoPARPs are important regulators of the immune response³-4 and are implicated in human diseases such as inflammation⁵ and cancer⁶-7, therefore small molecules that modulate the enzymatic activity of monoPARPs can be useful therapeutics. Despite interest in the development of monoPARP inhibitors, the field is lacking effective high-throughput assays that can be used to screen for and characterize modulators of monoPARP function. Thus, there is an urgent need for high-throughput assays that can be used to screen monoPARP modulators.

### SUMMARY

The present disclosure is related to methods of identifying PARP modulators.

The present invention is directed to a method of identifying an inhibitor for PARPs, the method comprising:
combining (i) a polypeptide comprising a PARP catalytic domain wherein the polypeptide is labeled with a donor fluorophore, (ii) a PARP probe, wherein the PARP probe is labeled with an acceptor fluorophore, and (iii) a test compound;
exposing the donor fluorophore to excitation light;
measuring a signal produced by the acceptor fluorophore; and
identifying the test compound as an inhibitor for PARP based on the signal produced by the acceptor fluorophore.

The present invention is further directed to a method of identifying an inhibitor for PARPs, the method comprising:
combining (i) a polypeptide comprising a PARP catalytic domain, wherein the polypeptide is labeled with an acceptor fluorophore; (ii) a PARP probe, wherein the PARP probe is labeled with a donor fluorophore, and (iii) a test compound;
exposing the donor fluorophore to excitation light;
measuring a signal produced by the acceptor fluorophore in the presence of a test compound; and
identifying the test compound as an inhibitor for PARP based on the signal produced by the acceptor fluorophore.

The present invention is further directed to a method of identifying an inhibitor for PARPs, the method comprising:
contacting a polypeptide comprising a PARP catalytic domain with a PARP probe in the presence of a test compound, wherein the PARP probe comprises a fluorophore;
exposing the probe to polarized excitation light, thereby generating fluorescence;
determining a fluorescence polarization value of the fluorescence; and
identifying the test compound as an inhibitor for PARP based on the fluorescence polarization value of the fluorescence.

The present invention is further directed to a method of identifying an inhibitor for PARPs, the method comprising:
contacting a fusion polypeptide with a PARP probe that comprises a fluorophore, wherein the fusion polypeptide comprises a PARP catalytic domain and a luciferase enzyme;
contacting the luciferase enzyme with a substrate to produce light, wherein the light can excite the fluorophore;
measuring a signal produced by the fluorophore in the presence of a test compound; and
identifying the test compound as an inhibitor for PARP based on the signal produced by the fluorophore.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

The patent or application file contains at least one drawing executed in color. Copies of this patent or patent application publication with color drawing(s) will be provided by the Office upon request and payment of the necessary fee.

FIG. 1 is a schematic diagram illustrating an example of in vitro TR-FRET probe displacement assay. An inhibitor will outcompete the biotinylated probe and decrease TR-FRET signal.

FIG. 2 is a schematic diagram illustrating an example of in vitro fluorescence polarization probe displacement assay. An inhibitor will outcompete the fluorophore-labeled probe and decrease fluorescence polarization signal.

FIG. 3 is a schematic diagram illustrating an example of bioluminescence resonance energy transfer probe displacement assay. An inhibitor will outcompete the fluorophore-labeled probe and decrease NanoBRET signal.

FIGS. 4A-4F. Validation results of the in vitro probe displacement binding assays. Dose response curves for Compound A were generated using each assay to confirm that Probe C was outcompeted from the monoPARP enzyme. IC₅₀ values for Compound A were 7 nM (TIPARP), 80 nM (PARP10), 200 nM (PARP12), 50 nM (PARP14), 60 nM (PARP15) and 100 nM (PARP16).

FIGS. 5A-5D. Validation results of the bioluminescence resonance energy transfer probe displacement binding assays. Dose response curves for control compounds were generated using each assay to confirm that Probe A was outcompeted from the monoPARP enzyme. IC₅₀ values for catalytic TIPARP Compound B=7 nM, full-length TIPARP Compound B=4 nM, PARP10 Compound C=4 nM, PARP12 Compound B=170 nM, PARP14 Compound A=30 nM.

FIGS. 6A-6F show the amino acid sequences of PARPs.

### DETAILED DESCRIPTION

PARPs refers to a family of proteins involved in a number of cellular processes such as DNA repair, genomic stability, and programmed cell death.

The primary function of PARPs is to post-translationally modify target proteins with ADP-ribose using NAD⁺ as substrate. The four best-studied family members, PARP1 and PARP5a along with their close functional homologs PARP2 and PARP5b respectively, all generate poly(ADP-ribose) (PAR). The main role of these PARPs is to detect and initiate an immediate cellular response to metabolic, chemical, or radiation-induced single-strand DNA breaks (SSB) by signaling the enzymatic machinery involved in the SSB repair. Once PARP detects a SSB, it binds to the DNA, undergoes a structural change, and begins the synthesis of a polymeric adenosine diphosphate ribose (poly (ADP-ribose)) chain, which acts as a signal for the other DNA-repairing enzymes.

However, many other PARPs do not generate PAR, and instead attach ADP-ribose as a monomer ADP-ribose (MAR) onto target proteins. Recent data has shown that many of these MAR-generating PARPs (monoPARPs) can have cancer relevant functions or inflammation related functions. MonoPARP enzymes have structurally related active sites which bind to nicotinamide adenine dinucleotide (NAD⁺) and catalyze the transfer of adenosine diphosphoribose to a substrate amino acid. Enzyme-linked immunosorbent assays (ELISA) measuring the incorporation of biotin-NAD⁺ to histones or to the monoPARP itself in an automodification reaction have been used to screen for monoPARP modulators⁸. These reactions are not catalytically efficient, and high concentrations of enzyme are needed to generate sufficient turnover to be detected. Since the lowest measurable IC₅₀ in an enzyme assay is half of the total enzyme concentration⁹, these assays are usually unable to differentiate and rank order very potent compounds. Thermal shift assays (TSA) have also been used to screen inhibitors of monoPARPs¹⁰, however these assays consume large amounts of protein and are at best semi-quantitative since compounds with similar binding affinities can have different effects on protein stabilization¹¹.

The present disclosure provides a more effective way to identify PARP modulators, and provides a series of high affinity active site probes that bind in the NAD⁺ pocket of monoPARPs which can be used to develop high-throughput biophysical assays.

### Poly (ADP-ribose) polymerases

There are 17 PARPs. The enzymatic activity and cancer relevant functions of these PARPs are summarized in Table 1 below.

TABLE 1

| | Other | | | | Cancer Related | |
|---|---|---|---|---|---|---|
| PARP | Names | Activity | NCBI Accession No. | Catalytic Domain | Functions | Cancers to target |
| PARP1 | ARTD1 | PAR | NP_001609.2 | Amino acids | DNA Repair, | HR deficient |
| | | | (SEQ ID NO: 13) | 788-1014 of | ERK/ NF-kB | Elevated |
| | | | | accession no. | signaling, Heat | ERK/NF-kB |
| | | | | NP_001609.2 | shock response | signaling |
| PARP2 | ARTD2 | PAR | NP_005475.2 | Amino acids | DNA Repair | HR deficient |
| | | | (SEQ ID NO: 14) | 356-583 of | | |
| | | | | accession no. | | |
| | | | | NP_005475.2 | | |
| PARP3 | ARTD3 | MAR | NP_001003931.3 | Amino acids | DNA Repair | DNA repair |
| | | | (SEQ ID NO: 15) | 313-533 of | | deficient |
| | | | | accession no. | | |
| | | | | NP_001003931.3 | | |
| PARP4 | vPARP | MAR | NP_006428.2 | Amino acids | | |
| | ARTD4 | | (SEQ ID NO: 16) | 369-573 of | | |
| | | | | accession no. | | |
| | | | | NP_006428.2 | | |
| PARP5a | TNKS1 | PAR | NP_003738.2 | Amino acids | Telomere | Elevated Wnt |
| | ARTD5 | | (SEQ ID NO: 17) | 1112-1317 of | Maintenance, | Signaling |
| | | | | accession no. | Wnt Signaling, | Telomerase |
| | | | | NP_003738.2 | Proteasome | Dependent |
| | | | | | Regulation, | Stress Granule |
| | | | | | Stress Granule | Positive Solid |
| | | | | | Assembly, Cell | Tumors |
| | | | | | Division | |
| PARP5b | TNKS2 | PAR | NP_079511.1 | Amino acids | Telomere | Elevated Wnt |
| | ARTD6 | | (SEQ ID NO: 18) | 959-1164 of | Maintenance, | Signaling, |
| | | | | accession no. | Wnt Signaling | Telomerase |
| | | | | NP_079511.1 | | Dependent |
| PARP6 | ARTD17 | MAR | NP_001310451.1 | Amino acids | Negative | Potential |
| | | | (SEQ ID NO: 19) | 394-620 of | Regulator of | Tumor |
| | | | | accession no. | Proliferation | Suppressive |
| | | | | NP_001310451.1 | | Functions |
| TIPARP | PARP7 | MAR | NP_056323.2 | Amino acids | | |
| | ARTD14 | | (SEQ ID NO: 1) | 449-657 of | | |
| | | | | accession no. | | |
| | | | | NP_056323.2 | | |
| PARP8 | ARTD16 | MAR | AAH37386.1 | Amino acids | | |
| | | | (SEQ ID NO: 20) | 328-494 of | | |
| | | | | accession no. | | |
| | | | | AAH37386.1 | | |
| PARP9 | BAL1 | MAR | AAH39580.1 | Amino acids | Cell Migration | Metastatic |
| | ARTD9 | | (SEQ ID NO: 21) | 628-850 of | | Cancers |
| | | | | accession no. | | |
| | | | | AAH39580.1 | | |
| PARP10 | ARTD10 | MAR | NP_116178.2 | Amino acids | Inhibits | Potential |
| | | | (SEQ ID NO: 2) | 806-1025 of | Myc and NF-kB | Tumor |
| | | | | accession no. | signaling | Suppressive |
| | | | | NP_116178.2 | Pro-apoptotic | Functions |
| PARP11 | ARTD11 | MAR | Q9NR21.2 | Amino acids | | |
| | | | (SEQ ID NO: 22) | 123-338 of | | |
| | | | | accession no. | | |
| | | | | Q9NR21.2 | | |
| PARP12 | ARTD12 | MAR | NP_073587.1 | Amino acids | Stress Granule | Stress Granule |
| | | | (SEQ ID NO: 3) | 484-698 of | Assembly | Positive Solid |
| | | | | accession no. | | Tumors |
| | | | | NP_073587.1 | | |
| PARP13 | ZAP | MAR | NP_064504.2 | Amino acids | Stress Granule | Stress Granule |
| | ZC3HAV1 | | (SEQ ID NO: 23) | 716-902 of | Assembly, | Positive Solid |
| | ARTD13 | | | accession no. | miRNA-RISC | Tumors |
| | | | | NP_064504.2 | regulation | |
| PARP14 | BAL2 | MAR | NP_060024.2 | Amino acids | B cell survival, | Hematopoeitic |
| | ARTD8 | | (SEQ ID NO: 4) | 1605-1801 of | Cell Migration, | malignancies, |
| | | | | accession no. | Stress Granule | Metastatic |
| | | | | NP_060024.2 | Assembly | Cancers |
| PARP15 | BAL3 | MAR | NP_689828.1 | Amino acids | Stress Granule | Stress Granule |
| | ARTD7 | | (SEQ ID NO: 5) | 482-678 of | Assembly | Positive Solid |
| | | | | accession no. | | Tumors |
| | | | | NP_689828.1 | | |
| PARP16 | ARTD15 | MAR | NP_060321.3 | Amino acids | ER Unfolded | UPR |
| | | | (SEQ ID NO: 6) | 94-279 of | Protein | dependent |
| | | | | accession no. | Response | |
| | | | | NP_060321.3 | | |

Among these enzymes, PARP1, PARP2, PARP5a and PARP5b can generate poly (ADP-ribose) (PAR). PARP3, PARP4, PARP6, TIPARP, PARP8, PARP9, PARP10, PARP11, PARP12, PARP13, PARP14, PARP15, and PARP16 are monoPARPs.

PARPs have multiple diverse functions in physiological pathways including cell migration, transcriptional regulation, signal transduction, miRNA-mediated gene silencing, regulation of membrane organelles and telomere length regulation. Additionally, PARPs function in stress-responsive cellular pathways upon DNA damage, cytoplasmic stress, environmental stress and ER stress, activating DNA damage repair, stress granule assembly, the heat shock response and the ER unfolded protein response pathways in response. Many of these physiological and stress response pathways are misregulated in cancer or inflammation.

Thus, the inhibitors of PARPs (e.g., monoPARPs) can have various uses. For example, they can be used to modulate (e.g., inhibit or facilitate) cell migration, transcriptional regulation, signal transduction, and gene silencing. They can also be used to modulate (e.g., inhibit or facilitate) stress-responsive cellular pathways (e.g., upon DNA damage), or DNA damage repair pathways. In some embodiments, PARP inhibitors (e.g., monoPARP inhibitors) can be used to treat a disorder associated with PARP overexpression or overactivity. In some embodiments, PARP inhibitors (e.g., monoPARP inhibitors) can be used to treat cancers or inflammation.

A detailed description of PARPs and their functions can be found, e.g., in Vyas et al., “New PARP targets for cancer therapy,” Nature Reviews Cancer 14.7 (2014): 502, which is incorporated herein by reference in its entirety.

The present disclosure provides methods of identifying PARP modulators, and also provides polypeptides (e.g., fusion polypeptides) comprising a catalytic domain of PARPs (e.g., PARP1, PARP2, PARP3, PARP4, PARP5a, PARP5b, PARP6, TIPARP, PARP8, PARP9, PARP10, PARP11, PARP12, PARP13, PARP14, PARP15, or PARP16). These polypeptides can be used in various assays for identifying the modulators of interest (e.g., PARP inhibitors). As used herein, the term “catalytic domain” refers to a portion of an enzyme that has a catalytic activity. The catalytic domain comprises the region of an enzyme that interacts with its substrate to cause the enzymatic reaction. In many cases, the active site is located in the catalytic domain, and the substrate binds to active site.

In some embodiments, the catalytic domain is the catalytic domain of TIPARP (e.g., residues 449-657 of NP_056323.2 (SEQ ID NO: 1)), the catalytic domain of PARP10 (e.g., residues 806-1025 of NP_116178.2 (SEQ ID NO: 2)), the catalytic domain of PARP12 (e.g., residues 484-698 of NP_073587.1 (SEQ ID NO: 3)), the catalytic domain of PARP14 (e.g., residues 1605-1801 of NP_060024.2 (SEQ ID NO: 4)), the catalytic domain of PARP15 (e.g., residues 482-678 of NP_689828.1 (SEQ ID NO: 5)), or the catalytic domain of PARP16 (e.g., residues 5-279 of NP_060321.3 (SEQ ID NO: 6)).

In some embodiments, the catalytic domain is the catalytic domain of PARP1 (e.g., residues 788-1014 of NP_001609.2 (SEQ ID NO: 13)), the catalytic domain of PARP2 (e.g., residues 356-583 of NP_005475.2 (SEQ ID NO: 14)), the catalytic domain of PARP3 (e.g., residues 313-533 of NP_001003931.3 (SEQ ID NO: 15)), the catalytic domain of PARP4 (e.g., residues 369-573 of NP_006428.2 (SEQ ID NO: 16)), the catalytic domain of PARP5a (e.g., residues 1112-1317 of NP_003738.2 (SEQ ID NO: 17)), the catalytic domain of PARP5b (e.g., residues 959-1164 of NP_079511.1 (SEQ ID NO: 18)), the catalytic domain of PARP6 (e.g., residues 394-620 of NP 001310451.1 (SEQ ID NO: 19)), the catalytic domain of PARP8 (e.g., residues 328-494 of AAH37386.1 (SEQ ID NO: 20)), the catalytic domain of PARP9 (e.g., residues 628-850 of AAH39580.1 (SEQ ID NO: 21)), the catalytic domain of PARP11 (e.g., residues 123-338 of Q9NR21.2 (SEQ ID NO: 22)), or the catalytic domain of PARP13 (e.g., residues 716-902 of NP_064504.2 (SEQ ID NO: 23)).

In some embodiments, the polypeptide comprises a catalytic domain that has a sequence that is at least 85%, 90%, 91%, 92%, 93%, 94%, 95%, 96%, 97%, 98%, 99% identical to the sequence of a catalytic domain as described herein.

In some embodiments, the polypeptide (e.g., fusion polypeptide) comprises a region of a PARP, wherein the substrate binding pocket is located in this region of the PARP. In some embodiments, the polypeptide comprises residues 456-657 of NP_056323.2 (SEQ ID NO: 1), residues 808-1025 of NP_116178.2 (SEQ ID NO: 2), residues 489-684 of NP_073587.1 (SEQ ID NO: 3), residues 1611-1801 of NP_060024.2 (SEQ ID NO: 4), residues 481-678 of NP_689828.1 (SEQ ID NO: 5), or residues 5-279 of NP 060321.3 (SEQ ID NO: 6).

In some embodiments, the polypeptide can be linked with a label (e.g., a fluorophore). As used herein, the term “linked” refers to being covalently or non-covalently associated, e.g., by a chemical bond (e.g., a peptide bond, or a carbon-carbon bond), by hydrophobic interaction, by Van der Waals interaction, and/or by electrostatic interaction.

The label can be a chemical or composition detectable by spectroscopic, photochemical, biochemical, immunochemical, chemical, or other physical means. For example, useful labels include fluorescent dyes (fluorophores), luminescent agents, electron-dense reagents, enzymes (e.g., as commonly used in an ELISA, or luciferase), biotin, enzymes acting on a substrate (e.g., horseradish peroxidase), digoxigenin, ³²P and other isotopes, haptens, and proteins which can be made detectable, e.g., by incorporating a radiolabel into the peptide or used to detect antibodies specifically reactive with the peptide. The term includes combinations of single labeling agents, e.g., a combination of fluorophores that provides a unique detectable signature, e.g., at a particular wavelength or combination of wavelengths. Any method known in the art for conjugating label to a desired agent may be employed.

In some embodiments, the polypeptide can have a fusion tag (e.g., SEQ ID NO: 7, 8, 9, or 10). These fusion tags can be used to purify polypeptides.

In some embodiments, the polypeptide can have an epitope (e.g., 6xHis) that can be specifically recognized by an antibody (e.g., anti-6xHis antibody). A label (e.g., fluorophore) can be conjugated to the antibody, thereby associating with the polypeptide.

In some embodiments, the polypeptide is a fusion polypeptide and can comprise a luciferase (e.g., SEQ ID NO: 11). The luciferase can be located at the N-terminus or the C-terminus of the polypeptide.

The disclosure also provides a nucleic acid sequence that is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any nucleotide sequence as described herein, and an amino acid sequence that is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any amino acid sequence as described herein. In some embodiments, the nucleic acid sequence is at least 60%, 65%, 70%, 75%, 80%, 85%, 90%, 91%, 92%, 93%, 94%, 95%, 96%, 97%, 98%, 99% identical to any nucleotide sequence as described herein. In some embodiments, the amino acid sequence is at least 60%, 65%, 70%, 75%, 80%, 85%, 90%, 91%, 92%, 93%, 94%, 95%, 96%, 97%, 98%, 99% identical to any amino acid sequence as described herein.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The length of a reference sequence aligned for comparison purposes is at least 80% of the length of the reference sequence, and in some embodiments is at least 90%, 95%, or 100%. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences. For purposes of the present disclosure, the comparison of sequences and determination of percent identity between two sequences can be accomplished using a Blossum 62 scoring matrix with a gap penalty of 12, a gap extend penalty of 4, and a frameshift gap penalty of 5.

In some embodiments, the disclosure relates to nucleotide sequences encoding any peptides that are described herein, or any amino acid sequences that are encoded by any nucleotide sequences as described herein. In some embodiments, the nucleic acid sequence is less than 10, 20, 30, 40, 50, 60, 70, 80, 90, 100, 110, 120, 130, 150, 200, 250, 300, 350, 400, 500, 600, 700, 800, 900, or 1000 nucleotides. In some embodiments, the amino acid sequence is less than 5, 6, 7, 8, 9, 10, 20, 30, 40, 50, 60, 70, 80, 90, 100, 110, 120, 130, 140, 150, 160, 170, 180, 190, 200, 400, 500, 600, 700, 800, 900, or 1000 amino acid residues.

### PARP Probes

The present disclosure provides high affinity active site probes that bind in the NAD⁺ pocket of PARPs (e.g., monoPARPs) which can be used to develop high-throughput biophysical assays. These probes are based on the structure-activity relationship (SAR) and binding mode of inhibitors that bind to the NAD⁺ pocket of monoPARP enzymes. Thus, as used herein, the term “PARP probe” refers to an agent (e.g., a small molecule) that can bind to the active site of a PARP.

Many enzyme inhibitors inhibit the functions of enzymes by preventing a substrate from entering the enzyme's active site. Thus, if a test compound can prevent an active site probe from binding to the enzyme's active site, the test compound can also prevent a substrate from entering the enzyme's active site, thus working as an inhibitor. Therefore, in some embodiments, these PARP inhibitors can compete with PARP probes, bind to or occupy enzyme's active site, and/or displace the PARP probes.

In some embodiments, the PARP probe has a structure according to Formula (I):

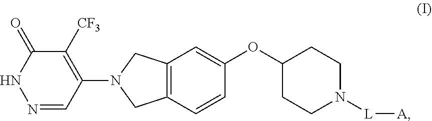

or a salt thereof,
wherein:
   L is a linking group having 10-30 spacer atoms selected from C, N, O, and S connecting the N atom of the piperidinyl group of Formula (I) with group A; and
   A is a fluorophore or an affinity tag.

In some embodiments,
L is:

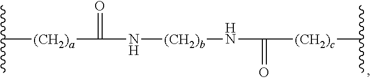

wherein:
a is 0, 1, or 2;
b is 1-26; and
c is 0, 1, or 2;
wherein the sum of a+b+c is 1 to 26, or
L is a chain of 5-30 atoms in length comprising—(CH₂CH₂O)d—wherein d is 2-10.

In some embodiments, A is an organic dye.

In some embodiments, A is biotin.

In some embodiments, A is:

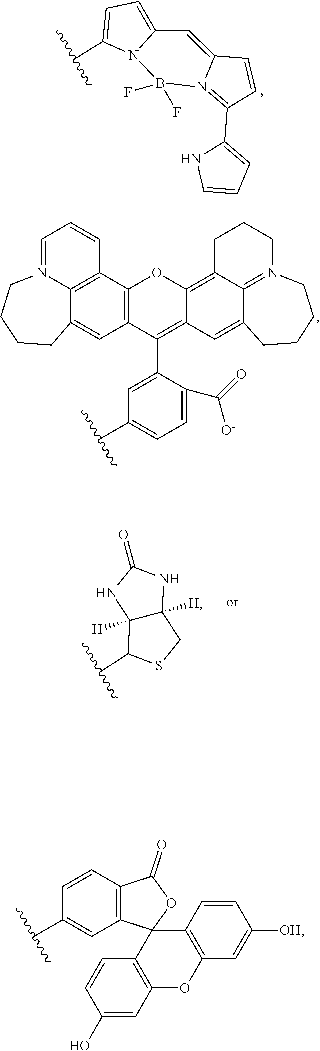

or a salt thereof.

As used herein, the term “fluorophore” refers to a chemical compound that can re-emit light upon light excitation. The donor is the fluorophore that emits light of shorter wavelength which is used to excite the acceptor, causing it to emit light of longer wavelength. Fluorophores can be organic molecules like fluorescein and similar organic dye moieties. Fluorophores can also include inorganic components like transition metals.

An affinity tag is a chemical or polypeptide group that can bind to other chemical or polypeptide molecules covalently or non-covalently (e.g., preferably with high affinity). Examples of non-covalent affinity tags are biotin which binds to streptavidin and antibodies, hexahistidine which binds to nickel-nitrilotriacetic acid and antibodies, glutathione which binds to glutathione S-transferase and antibodies, etc. Examples of covalent affinity tags are primary amines such as lysine which react with N-hydroxysuccinamide, as well as free thiols such as cysteine which react with other free thiols to form disulfide bonds. In some embodiments, the affinity tag is biotin.

Where a PARP probe “is labeled” with a fluorophore, the PARP probe can contain an affinity tag that covalently or non-covalently binds with a molecule (e.g., streptavidin or an antibody) having a fluorophore. Where a PARP probe comprises a fluorophore or an affinity tag, the fluorophore or affinity tag is generally understood to be part of the probe molecule.

The PARP probes of the invention, including salts thereof, can be prepared using known organic synthesis techniques and can be synthesized according to any of numerous possible synthetic routes.

The reactions for preparing compounds as described herein can be carried out in suitable solvents which can be readily selected by one of skill in the art of organic synthesis. Suitable solvents can be substantially nonreactive with the starting materials (reactants), the intermediates, or products at the temperatures at which the reactions are carried out, e.g., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected by the skilled artisan.

Preparation of compounds as described herein can involve the protection and deprotection of various chemical groups. The need for protection and deprotection, and the selection of appropriate protecting groups, can be readily determined by one skilled in the art. The chemistry of protecting groups can be found, for example, in T.W. Greene and P.G.M. Wuts, Protective Groups in Organic Synthesis, 3rd. Ed., Wiley & Sons, Inc., New York (1999), which is incorporated herein by reference in its entirety.

Reactions can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), or mass spectrometry, or by chromatography such as high performance liquid chromatography (HPLC) or thin layer chromatography.

The expressions, “ambient temperature,” “room temperature,” and “r.t.”, as used herein, are understood in the art, and refer generally to a temperature, e.g. a reaction temperature, that is about the temperature of the room in which the reaction is carried out, for example, a temperature from about 20° C. to about 30° C.

### Time-resolved Fluorescence Resonance Energy Transfer (TR-FRET) Probe Displacement Assay

The PARP probes can be used in time-resolved fluorescence resonance energy transfer (TR-FRET) probe displacement assays. TR-FRET is the combination of time-resolved fluorometry (TRF) with Förster resonance energy transfer (FRET). It can offer a powerful tool for studying the interactions between biomolecules.

FRET occurs when a donor fluorophore in its excited state transfers energy by a non-radiative dipole-dipole coupling mechanism to an acceptor fluorophore in close proximity (e.g., <10 nm). As a result, the acceptor emission is predominantly observed because of the intermolecular FRET from the donor to the acceptor. FRET can be quantified in cuvette-based experiments or in microscopy images on a pixel-by-pixel basis. This quantification can be based directly on detecting two emission channels under two different excitation conditions (primarily donor and primarily acceptor). However, for robustness reasons, FRET quantification is most often based on measuring changes in fluorescence intensity.

The biological fluids or serum commonly used in these research applications contain many compounds and proteins which are naturally fluorescent. Therefore, the use of conventional, steady-state fluorescence measurement presents serious limitations in assay sensitivity.

To reduce assay interference and increase data quality, a time-resolved FRET (TR-FRET) assay can be used to identify PARP inhibitors. TR-FRET generally employs a long-lifetime donor fluorophore (e.g., terbium chelate, samarium, europium, terbium, and dysprosium) and a suitable acceptor fluorophore (fluorescein or allophycocyanin).

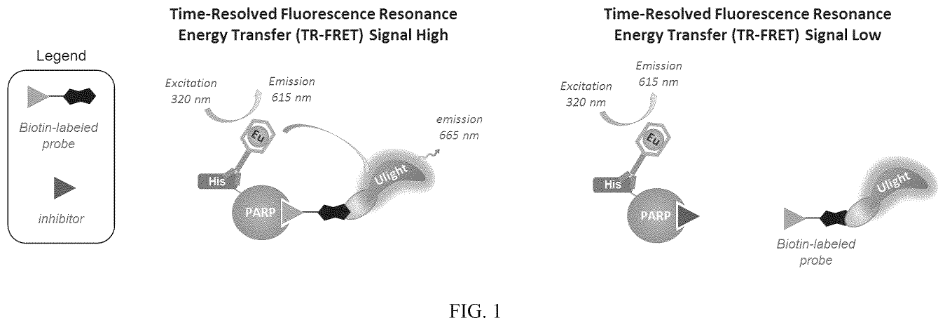

As shown in FIG. 1, TR-FRET can be used to identify an agent that can inhibit the binding between the PARP probe and the PARP polypeptide. The methods involve providing a polypeptide comprising a PARP catalytic domain. The polypeptide is labeled with a donor fluorophore. In the meantime, the PARP probe is labeled with an acceptor fluorophore. When the donor fluorophore is exposed to excitation light, the energy is transferred from the donor fluorophore to the acceptor fluorophore. The signal produced by the acceptor fluorophore is measured. In the presence of an agent (e.g., a PARP inhibitor) that can inhibit the binding between the PARP probe and the PARP polypeptide, the PARP probe is not in close proximity with the PARP polypeptide, thus the energy cannot be effectively transferred from the donor fluorophore to the acceptor fluorophore. Therefore, the signal produced by the acceptor fluorophore will decrease.

In certain embodiments, an agent (e.g., a test compound) is identified as a PARP inhibitor if it results in a decrease in the signal produced by the acceptor fluorophore by at least 10%, at least 15%, at least 20%, at least 25%, at least 30%, at least 40%, at least 50%, at least 60%, at least 70%, at least 80%, at least 90%, or at least 95% as compared to the signal of the acceptor fluorophore in the absence of the agent or any agent that can inhibit or interfere with the binding between the PARP probe and the PARP polypeptide.

In some embodiments, the signal produced by the acceptor fluorophore can be compared to a reference level. The reference level can be the signal produced by the acceptor fluorophore in the absence of any agent that can inhibit or interfere with the binding between the PARP probe and the polypeptide.

In certain embodiments, an agent (e.g., a test compound) is identified as a PARP inhibitor if it results in a decrease in the ratio of the signal produced by the acceptor to the signal produced by the donor by at least 10%, at least 15%, at least 20%, at least 25%, at least 30%, at least 40%, at least 50%, at least 60%, at least 70%, at least 80%, at least 90%, or at least 95% as compared to the ratio in the absence of the agent or any agent that can inhibit or interfere with the binding between the PARP probe and the PARP polypeptide.

In some embodiments, the ratio of the signal produced by the acceptor to the signal produced by the donor can be compared to a reference level. The reference level can be the ratio of the acceptor signal to the donor signal in the absence of any agent that can inhibit or interfere with the binding between the PARP probe and the polypeptide.

In some embodiments, the polypeptide can be labeled with a donor fluorophore, and the PARP probe can be labeled with an acceptor fluorophore.

A variety of fluorophore combinations can be used in TR-FRET. In some embodiments, lanthanide ion complexes (Ln (III) chelates or cryptates) are used. In some embodiments, the donor fluorophore is Europium³⁺, and the acceptor fluorophore is allophycocyanin. In some embodiments, the donor fluorophore is Terbium³⁺, and the acceptor fluorophore is phycoerythrin.

TR-FRET measurements can be also carried out using any suitable technique. For example, a microscope image of donor emission can be taken with the acceptor being present. The acceptor is then bleached, such that it is incapable of accepting energy transfer and another donor emission image is acquired. A pixel based quantification using the second equation in the theory section above is then possible. An alternative way of temporarily deactivating the acceptor is based on its fluorescence saturation.

In some embodiments, the ratio between the signal produced by the acceptor and the signal produced by the donor is calculated. The % inhibition can be calculated as described below:

$$\% inhibition = 100 \times \frac{{TRF}_{cmpd} - {TRF}_{min}}{{TRF}_{mαx} - {TRF}_{min}}$$

wherein TRF_{cmpd} is the TR-FRET ratio from the compound treated solution, TREₘᵢₙ is the TR-FRET ratio from a positive control and TRFₘₐₓ is the TR-FRET ratio from the negative control (e.g., DMSO-treated).

The % inhibition values can be plotted as a function of compound concentration and the following 4-parameter fit can be applied to derive the IC₅₀ values:

$$Y = Bottom + \frac{( Top - Bottom )}{( 1 + {( \frac{X}{{IC}_{50}} )}^{Hill Coefficient}}$$

wherein top and bottom can be normally allowed to float, but may be fixed at 100 or 0 respectively in a 3-parameter fit. The Hill Coefficient can be normally allowed to float but may also be fixed at 1 in a 3-parameter fit. Y is the % inhibition and X is the compound concentration. IC₅₀ is the concentration of an inhibitor where the response (or binding) is reduced by half. It is a measure of the potency of a substance in inhibiting a specific biological or biochemical function. Based on the modeling, IC₅₀ can be estimated.

FRET and TR-FRET methods, protocols, techniques, and assays are described generally and specifically in a number of patents and patent applications, including, e.g., U.S. Pat. Nos. 6,908,769; 6,824,990; 6,762,280; 6,689,574; 6,661,909; 6,642,001; 6,639,078; 6,472,156; 6,456,734; 6,376,257; 6,348,322; 6,323,039; 6,291,201; 6,280,981; 5,914,245; 5,661,035; and US 20080113444; US 2009021510; Du et al. “A time-resolved fluorescence resonance energy transfer assay for high-throughput screening of 14-3-3 protein-protein interaction inhibitors.” Assay and drug development technologies 11.6 (2013): 367-381; each of which is incorporated herein by reference in its entirety.

### Fluorescence Polarization Probe Displacement Assay

The PARP probes can also be used in a fluorescence polarization probe displacement assay. Fluorescence polarization (FP) is a homogeneous method that allows rapid and quantitative analysis of diverse molecular interactions and enzyme activities. This technique has been widely used in clinical and biomedical settings, and high-throughput screening (HTS).

In fluorescence polarization assays, a fluorophore is excited with polarized excitation light; the polarized fluorescence is then measured through an emission polarizer either parallel or perpendicular to the exciting light's plane of polarization. If a fluorescent molecule is stationary and exposed to plane-polarized light, it will become excited and consequently emit radiation back to the polarized-plane. However, if the excited fluorescent molecule is in motion (rotational or translational) during the fluorescent lifetime, it will emit light in a different direction than the excitation plane. The rate at which a molecule rotates is indicative of its size. When a fluorescent-labelled molecule binds to another molecule, the rotational motion will change, resulting in an altered intensity of plane-polarized light, which results in altered fluorescence polarization.

As shown in FIG. 2, when the PARP probe binds to the PARP polypeptide, the complex has a relatively larger molecular size. When an agent (e.g., PARP inhibitor) inhibits the binding between the PARP probe and the PARP polypeptide, the PARP probe with the fluorophore will not bind to the PARP polypeptide, thus the probe can rotate more quickly, resulting in a decrease of fluorescence polarization. This change can be detected, thereby determining whether an agent is a PARP inhibitor. In certain embodiments, an agent (e.g., a test compound) is identified as a PARP inhibitor if the fluorescence polarization decreases by at least 10%, at least 15%, at least 20%, at least 25%, at least 30%, at least 40%, at least 50%, at least 60%, at least 70%, at least 80%, at least 90%, or at least 95% as compared to the fluorescence polarization in the absence of the agent or any agent that can inhibit or interfere with the binding between the PARP probe and the PARP polypeptide.

A detailed description of fluorescence polarization and the method of implementing it is described, e.g., in Lea, Wendy A., and Anton Simeonov. “Fluorescence polarization assays in small molecule screening.” Expert opinion on drug discovery 6.1 (2011): 17-32, U.S. Pat. No. 6,432,632; US20030082665; each of which is incorporated herein by reference in its entirety.

### Bioluminescence Resonance Energy Transfer (BRET) Probe Displacement Assay

The PARP probes can also be used in bioluminescence resonance energy transfer (BRET) probe displacement assays. A limitation of FRET is the requirement for external illumination to initiate the fluorescence transfer, which can lead to background noise. Bioluminescence resonance energy transfer involves a bioluminescent luciferase (e.g., the luciferase from Renilla reniformis, or Oplophorus gracilirostris) to produce an initial photon emission.

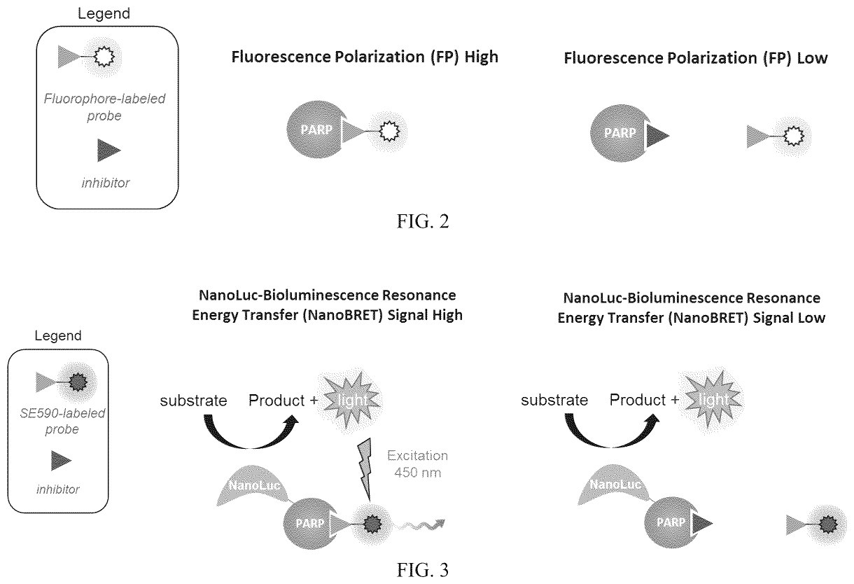

As compared to FRET, in BRET, the donor fluorophore is replaced by a luciferase. As shown in FIG. 3, in the presence of a substrate, bioluminescence from the luciferase excites the acceptor fluorophore through Förster resonance energy transfer mechanisms. Thus, if the acceptor fluorophore is in close proximity with the luciferase, the acceptor fluorophore accepts the energy from the luciferase and emits a light with different length.

When an agent (e.g., PARP inhibitor) inhibits the binding between PARP probe and the PARP polypeptide, the PARP probe with the acceptor fluorophore will not be in close proximity with the PARP polypeptide, thus the light from the luciferase enzyme reaction cannot be transferred to the acceptor fluorophore, resulting a decrease of fluorescence emitted from the acceptor fluorophore. This change can be detected, thereby determining whether an agent is a PARP inhibitor. In certain embodiments, an agent (e.g., a test compound) is identified as a PARP inhibitor if it results in a decrease in the fluorescence emitted from the acceptor fluorophore by at least 10%, at least 15%, at least 20%, at least 25%, at least 30%, at least 40%, at least 50%, at least 60%, at least 70%, at least 80%, at least 90%, or at least 95% as compared to the fluorescence emitted from the acceptor fluorophore in the absence of the agent or any agent that can inhibit or interfere with the binding between the PARP probe and the PARP polypeptide.

In some embodiments, the luciferase is NanoLuc, and the acceptor fluorophore is NanoBRET® 590SE.

BRET ratio can be measured by the formula as shown below:

$$BRET ratio = \frac{Emission of the acceptor fluorophore}{Luminescence}$$

Control wells containing a negative control or a positive control are used to calculate the % inhibition as described below:

$$\% inhibition = 100 \times \frac{BRET {ratio}_{cmpd} - BRET {ratio}_{min}}{BRET {ratio}_{max} - BRET {ratio}_{min}}$$

wherein BRET ratio_{cmpd} is the BRET ratio from the compound treated solution, BRET ratioₘᵢₙ is the BRET ratio from the positive control and BRET ratioₘₐₓ is the BRET ratio from the negative control.

The % inhibition values are plotted as a function of compound concentration and the following 4-parameter fit is applied to derive the IC₅₀ values:

$$Y = Bottom + \frac{( Top - Bottom )}{( 1 + {( \frac{X}{{IC}_{50}} )}^{Hill Coefficient}}$$

wherein top and bottom can be normally allowed to float, but may be fixed at 100 or 0 respectively in a 3-parameter fit. The Hill Coefficient can be normally allowed to float but may also be fixed at 1 in a 3-parameter fit. Y is the % inhibition and X is the compound concentration. The IC₅₀ value can be derived from the modeling.

### High Throughput Screening and Compound Library

The assays as described herein can be used in high throughput screening for PARP modulators (e.g., monoPARP inhibitors). Such assays can be used to screen small molecule libraries available from various commercial sources. Screening of such libraries, including combinatorially generated libraries (e.g., peptide libraries), is a rapid and efficient way to screen a large number of related (and unrelated) compounds for activity.

This disclosure provides methods for screening test compounds, e.g., polypeptides (including, e.g., antibodies and antigen-binding fragments thereof), polynucleotides, inorganic or organic large or small molecule test compounds, to identify agents useful for modulating PARP enzymatic activity, and for the treatment of disorders associated with PARP overexpression or overactivity (e.g., cancer or inflammation).

As used herein, “small molecules” refers to small organic or inorganic molecules of molecular weight below about 3,000 Daltons. In general, small molecules useful for the methods as described herein can have a molecular weight of less than 3,000 Daltons (Da). The small molecules can be, e.g., from at least about 100 Da to about 3,000 Da (e.g., between about 100 to about 3,000 Da, about 100 to about 2500 Da, about 100 to about 2,000 Da, about 100 to about 1,750 Da, about 100 to about 1,500 Da, about 100 to about 1,250 Da, about 100 to about 1,000 Da, about 100 to about 750 Da, about 100 to about 500 Da, about 200 to about 1500, about 500 to about 1000, about 300 to about 1000 Da, or about 100 to about 250 Da).

The test compounds can be, e.g., natural products or members of a combinatorial chemistry library. A set of diverse molecules should be used to cover a variety of functions such as charge, aromaticity, hydrogen bonding, flexibility, size, length of side chain, hydrophobicity, and rigidity. Combinatorial techniques suitable for synthesizing small molecules are known in the art, e.g., as exemplified by Obrecht and Villalgordo, Solid-Supported Combinatorial and Parallel Synthesis of Small-Molecular-Weight Compound Libraries, Pergamon-Elsevier Science Limited (1998), and include those such as the “split and pool” or “parallel” synthesis techniques, solid-phase and solution-phase techniques, and encoding techniques (see, for example, Czarnik, Curr. Opin. Chem. Bio. 1:60-6 (1997)). In addition, a number of small molecule libraries are commercially available.

Libraries screened using the methods as described herein can comprise a variety of types of test compounds. A given library can comprise a set of structurally related or unrelated test compounds. In some embodiments, the test compounds are peptide or peptidomimetic molecules. In some embodiments, the test compounds are nucleic acids. In some embodiments, the test compounds are antibodies or antigen-binding fragments thereof.

In some embodiments, the test compounds and libraries thereof can be obtained by systematically altering the structure of a first test compound, e.g., a first test compound that is structurally similar to a known natural binding partner of the target polypeptide, or a first small molecule identified as capable of binding the target polypeptide, e.g., using methods known in the art or the methods described herein, and correlating that structure to a resulting biological activity, e.g., a structure-activity relationship study. As one of skill in the art will appreciate, there are a variety of standard methods for creating such a structure-activity relationship. Thus, in some instances, the work may be largely empirical, and in others, the three-dimensional structure of an endogenous polypeptide or portion thereof can be used as a starting point for the rational design of a small molecule compound or compounds For example, in one embodiment, a general library of small molecules is screened, e.g., using the methods described herein.

In some embodiments, a test compound is applied to a test sample, e.g., a cell or living tissue, e.g., tumor tissue, and one or more effects of the test compound is evaluated. For example, the ability of the test compound to inhibit cell growth or tumor growth is evaluated.

In some embodiments, the test sample is, or is derived from (e.g., a sample taken from) an in vivo model. For example, an animal model, e.g., a rodent such as a rat, can be used.

Thus, test compounds identified as “hits” (e.g., test compounds that can inhibit PARP) in a first screen can be selected and systematically altered, e.g., using rational design, to optimize binding affinity, avidity, specificity, or other parameter. Such optimization can also be screened for using the methods described herein. Thus, in one embodiment, the disclosure includes screening a first library of compounds using a method known in the art and/or described herein, identifying one or more hits in that library, subjecting those hits to systematic structural alteration to create a second library of compounds structurally related to the hit, and screening the second library using the methods described herein.

Test compounds identified as hits can be considered candidate therapeutic compounds, useful in treating disorders associated with PARP overexpression or overactivity (e.g., cancer). A variety of techniques useful for determining the structures of “hits” can be used in the methods described herein, e.g., NMR, mass spectrometry, gas chromatography equipped with electron capture detectors, fluorescence and absorption spectroscopy. Thus, the disclosure also includes compounds identified as “hits” by the methods described herein, and methods for their administration and use in the treatment, prevention, or delay of development or progression of a disorder described herein.

Test compounds identified as candidate therapeutic compounds can be further screened by administration to an animal model of a disorder associated with PARP overexpression or overactivity (e.g., cancer). The animal can be monitored for a change in the disorder, e.g., for an improvement in a parameter of the disorder, e.g., a parameter related to clinical outcome. In some embodiments, the parameter is tumor growth, and an improvement would inhibit tumor growth.

In some embodiments, the PARP modulators obtained from the screening are inhibitors of PARP1, PARP2, PARP3, PARP4, PARP5a, PARP5b, PARP6, TIPARP, PARP8, PARP9, PARP10, PARP11, PARP12, PARP13, PARP14, PARP15, or PARP16. In some embodiments, the inhibitors have an IC₅₀ for a PARP (e.g., PARP1, PARP2, PARP3, PARP4, PARP5a, PARP5b, PARP6, TIPARP, PARP8, PARP9, PARP10, PARP11, PARP12, PARP13, PARP14, PARP15, or PARP16) of less than 1, 2, 3, 4, 5, 6, 7, 8, 9, 10, 20, 30, 40, 50, 60, 70, 80, 90, 100, 110, 120, 130, 140, 150, 160, 170, 180, 190, or 200 nM.

In some embodiments, the binding affinity of the PARP inhibitors (K_{d}) (between the compound and PARP) is less than 1×10⁻⁶ M, less than 1×10⁻⁷ M, less than 1×10⁻⁸ M, less than 1×10⁻⁹ M, or less than 1×10⁻¹⁰ M. In some embodiments, the Ka is less than 50 nM, 30 nM, 20 nM, 15 nM, 10 nM, 9 nM, 8 nM, 7 nM, 6 nM, 5 nM, 4 nM, 3 nM, 2 nM, or 1 nM. In some embodiments, Ka is greater than 1×10⁻⁷ M, greater than 1×10⁻⁸ M, greater than 1×10⁻⁹ M, greater than 1×10⁻¹⁰ M, greater than 1×10⁻¹¹ M, or greater than 1×10⁻¹² M.

### Methods of Treatment

The methods described herein include methods for the treatment of diseases sor disorders associated with PARP overexpression or overactivity comprising administering to a patient in need thereof a therapeutically effective amount of a compound identified according to one or more of the assays described herein. In some embodiments, the disease or disorder is cancer. Generally, the methods include administering a therapeutically effective amount of PARP modulators (e.g., inhibitors) identified by the methods as described herein, to a subject who is in need of, or who has been determined to be in need of, such treatment. As used in this context, to “treat” means to ameliorate at least one symptom of the disorder associated with PARP overexpression or overactivity. Often, the treatment results in a decreased activity of PARP.

In some embodiments, the disease or disorder is cancer In some embodiments, administration of a therapeutically effective amount of a compound described herein can result in a decrease of tumor size or tumor volume, a decrease of tumor growth, a reduction of the increase rate of tumor volume in a subject (e.g., as compared to the rate of increase in tumor volume in the same subject prior to treatment or in another subject without such treatment), a decrease in the risk of developing a metastasis or the risk of developing one or more additional metastasis, an increase of survival rate, and an increase of life expectancy, etc.

As used herein, the term “cancer” refers to cells having the capacity for autonomous growth, i.e., an abnormal state or condition characterized by rapidly proliferating cell growth. The term is meant to include all types of cancerous growths or oncogenic processes, metastatic tissues or malignantly transformed cells, tissues, or organs, irrespective of histopathologic type or stage of invasiveness. The term “tumor” as used herein refers to cancerous cells, e.g., a mass of cancerous cells.

As used herein, the terms “subject” and “patient” are used interchangeably throughout the specification and describe an animal, human or non-human, to whom treatment according to the methods of the present invention is provided. Veterinary and non-veterinary applications are contemplated by the present invention. Human patients can be adult humans or juvenile humans (e.g., humans below the age of 18 years old). In addition to humans, patients include but are not limited to mice, rats, hamsters, guinea-pigs, rabbits, ferrets, cats, dogs, and primates. Included are, for example, non-human primates (e.g., monkey, chimpanzee, gorilla, and the like), rodents (e.g., rats, mice, gerbils, hamsters, ferrets, rabbits), lagomorphs, swine (e.g., pig, miniature pig), equine, canine, feline, bovine, and other domestic, farm, and zoo animals.

### Kits and Compositions

The present disclosure also provides kits for PARP inhibitor screening. The kit can include a PARP probe comprising a fluorophore or an affinity tag. The kit can also include a polypeptide comprising a PARP catalytic domain and optionally a fusion/affinity tag (e.g., hexahistidine). In some embodiments, the polypeptide is labeled with a fluorophore.

The present disclosure also provides compositions for the PARP inhibitors. In some embodiments, the compositions are formulated with a pharmaceutically acceptable carrier. The pharmaceutical compositions and formulations can be administered parenterally, topically, orally or by local administration, such as by aerosol or transdermally. The pharmaceutical compositions can be formulated in any way and can be administered in a variety of unit dosage forms depending upon the condition or disease and the degree of illness, the general medical condition of each patient, the resulting preferred method of administration and the like. Details on techniques for formulation and administration of pharmaceuticals are well described in the scientific and patent literature, see, e.g., Remington: The Science and Practice of Pharmacy, 21st ed., 2005.

### EXAMPLES

The invention is further described in the following examples, which do not limit the scope of the invention described in the claims.

### Materials and Methods

The following equipment and methods were used in the following examples.

¹H NMR Spectra were recorded at 300 or 400 MHz using a Bruker AVANCE 400 MHz spectrometer. NMR interpretation was performed using MestReC or MestReNova software to assign chemical shift and multiplicity. In cases where two adjacent peaks of equal or unequal height were observed, these two peaks may be labeled as either a multiplet or as a doublet. In the case of a doublet, a coupling constant using this software may be assigned. In any given example, one or more protons may not be observed due to obscurity by water and/or solvent peaks.

Liquid chromatography—mass spectrometry (LCMS) equipment and conditions were as follows:
1. Liquid chromatography (LC): Agilent Technologies 1290 series, Binary Pump, Diode Array Detector. Agilent Poroshell 120 EC-C18, 2.7 μm, 4.6×50 mm column. Mobile phase: A: 0.05% Formic acid in water (v/v), B: 0.05% Formic acid in ACN (v/v). Flow Rate: 1 mL/min at 25° C. Detector: 214 nm, 254 nm. Gradient stop time, 10 min. Timetable is shown below:

TABLE 2

| T (min) | A (%) | B (%) |
|---|---|---|
| 0.0 | 90 | 10 |
| 0.5 | 90 | 10 |
| 8.0 | 10 | 90 |
| 10.0 | 0 | 100 |

2. Mass spectrometry (MS): G6120A, Quadrupole LC/MS, Ion Source: ES-API, TIC: 70˜1000 m/z, Fragmentor: 60, Drying gas flow: 10 L/min, Nebulizer pressure: 35 psi, Drying gas temperature: 350° C., Vcap: 3000V.
3. Sample preparation: samples were dissolved in ACN or methanol at 1˜10 mg/mL, then filtered through a 0.22 μm filter membrane. Injection volume: 1˜10.

The following abbreviations are used in the disclosure: ACN (acetonitrile); Boc (tert-butoxycarbonyl); Boc2O (di-tert-butyl dicarbonate); CuI (copper iodide); CDCl₃ (deuterated chloroform); CD₃OD (deuterated methanol); DCM (dichloromethane); DIPEA (N,N-diisopropylethylamine); DMF (N,N-dimethylformamide); DMSO (dimethylsulfoxide); DMSO-d₆ (deuterated dimethylsulfoxide); eq (equivalent); EtOAc (ethyl acetate); g (gram); h (hour); HATU (1-[bis(dimethylamino)methylene]-1H-1,2,3-triazolo[4,5-b]pyridinium 3-oxide hexafluorophosphate); ¹H NMR (proton nuclear magnetic resonance); HCl (hydrochloric acid); Hz (hertz); L (litre); LiCl (lithium chloride); LCMS (liquid chromatography-mass spectrometry); M (molar); MeOH (methanol); mg (milligrams); MHz (megahertz); min (minutes); mL (millilitres), mmol (millimoles); NMP (N-methyl-2-pyrrolidone); prep-HPLC (preparative high-performance liquid chromatography); Pd(OAc)2 (palladium (II) acetate); ppm (parts per million); Pd(allyl)Cl₂ (Bis(η3-allyl)di(μ-chloro)dipalladium(II)); Rockphos (2-Di(tert-butyl)phosphino-2,4,6-triisopropyl-3-methoxy-6-methylbiphenyl); RT (room temperature); SEM (2-(trimethylsilyl)ethoxymethyl); SEMC1 (2-(trimethylsilyl)ethoxymethyl chloride); TEA (triethyl amine); tBuBrettphos (2-(Di-tert-butylphosphino)-2,4,6-triisopropyl-3,6-dimethoxy-1,1-biphenyl); THF (tetrahydrofuran); TLC (thin layer chromatography); v/v (volume/volume).

### Example 1: Synthesis of Intermediates

### Int-A1: 5-Chloro-4-(trifluoromethyl)-2-[[2-(trimethylsilyl)ethoxy]methyl]-2,3-dihydropyridazin-3-one

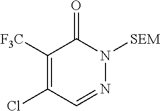 [SMILES: C[Si](C)(C)CCOCn1ncc(Cl)c(C(F)(F)F)c1=O]

### Step 1: 4,5-Dibromo-2-[[2-(trimethylsilyl)ethoxy]methyl]-2,3-dihydropyridazin-3-one

To a solution of 4,5-dibromo-2,3-dihydropyridazin-3-one (3500 g, 13.78 mol, 1.00 equiv) in DMF (30 L) was added sodium hydride (400 g, 16.56 mol, 1.20 equiv) in batches at 0° C. under nitrogen. The resulting solution was stirred for 1 hour at room temperature, then [2-(chloromethoxy)ethyl]trimethylsilane (2500 g, 15.2 mol, 1.10 equiv) was added dropwise at 0° C. and stirred for 2 hours at room temperature. The reaction was then quenched by the addition of 30 L of water. The resulting solution was extracted with 3×50 L of ethyl acetate and the organic layers combined. The organic layers were washed with 3×30 L of brine, dried over anhydrous sodium sulfate and concentrated under reduced pressure to afford 4.2 kg of title compound. LCMS: [M+H]⁺ 384.70.

### Step 2: 4-Bromo-5-chloro-2-[[2-(trimethylsilyl)ethoxy]methyl]-2,3-dihydropyridazin-3-one

To a solution of 4,5-dibromo-2-[[2-(trimethylsilyl)ethoxy]methyl]-2,3-dihydropyridazin-3-one (2200 g, 5.73 mol, 1.00 equiv) in NMP (6 L) was added chlorolithium (231 g, 5.73 mol, 1.00 equiv) and stirred for 4 hours at 95° C. The reaction was then diluted by the addition of 10 L of water, extracted with 3×20 L of ethyl acetate and the organic layers combined. The organic layers were washed with 3×20 L of brine, dried over anhydrous sodium sulfate and concentrated under reduced pressure. The residue was purified by column chromatography (EtOAc: petroleum ether, 1:50, v/v) to afford 4.2 kg of 4,5-dibromo-2-[[2-(trimethylsilyl)ethoxy]methyl]-2,3-dihydropyridazin-3-one. This was repeated 2 times resulting in 2.2 kg of 4-bromo-5-chloro-2-[[2-(trimethylsilyl)ethoxy]methyl]-2,3-dihydropyridazin-3-one. LCMS: [M+H]⁺ 340.90.

### Step 3: 5-Chloro-4-(trifluoromethyl)-2-[[2-(trimethylsilyl)ethoxy]methyl]-2,3-dihydropyridazin-3-one

To a solution of 4-bromo-5-chloro-2-[[2-(trimethylsilyl)ethoxy]methyl]-2,3-dihydropyridazin-3-one (1100 g, 3.23 mol, 1.00 equiv) in NMP (6 L) at room temperature was added CuI (56 g, 0.64 mol, 0.20 equiv) followed by dropwise addition of methyl 2,2-difluoro-2-(fluorosulfonyl)acetate (1865 g, 9.7 mol, 3.00 equiv). The resulting solution was stirred for 2 hours at 80° C. The reaction was then quenched by the addition of 10 L of water and extracted with 3×10 L of ethyl acetate. The organic layers were combined and washed with 3×10 L of brine, dried over anhydrous sodium sulfate, and concentrated under reduced pressure. The residue was purified by column chromatography (ethyl acetate/petroleum ether, 1/100, v/v) to afford 1030 g (76%) of the title compound. LCMS: [M+H]⁺ 329.00.

¹H NMR (300 MHz, CDCl₃) δ 7.82 (s, 1H), 5.50 (d, J=27.3 Hz, 2H), 3.74 (dt, J=12.9, 8.2 Hz, 2H), 0.97 (td, J=8.3, 5.0 Hz, 2H), 0.01 (d, J=2.1 Hz, 9H).

### Int-A2: 2-[4-([2-[6-Oxo-5-(trifluoromethyl)-1,6-dihydropyridazin-4-yl]-2,3-dihydro-1H-isoindol-5-yl]oxy)piperidin-1-yl]acetic acid

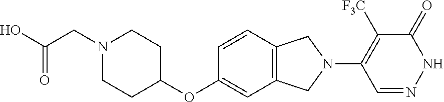 [SMILES: O=C(O)CN1CCC(Oc2ccc3c(c2)CN(c2cn[nH]c(=O)c2C(F)(F)F)C3)CC1]

### Step 1: 5-(5-Hydroxy-2,3-dihydro-1H-isoindol-2-yl)-4-(trifluoromethyl)-2-[[2-(trimethylsilyl)ethoxy]methyl]-2,3-dihydropyridazin-3-one

A solution of 5-chloro-4-(trifluoromethyl)-2-[[2-(trimethylsilyl)ethoxy]methyl]-2,3-dihydropyridazin-3-one (2.8 g, 8.52 mmol, 1.00 equiv), 2,3-dihydro-1H-isoindol-5-ol hydrobromide (4.27 g, 19.76 mmol, 1.00 equiv), and TEA (10 mL) in ethanol (40 mL) was stirred for 1 h at 60° C. The resulting solution was extracted with 2×100 mL of ethyl acetate and the organic layers combined and concentrated under reduced pressure to afford 4.5 g of the title compound as a yellow oil. LCMS: [M+H]⁺ 428.23.

### Step 2: tert-Butyl 4-([2-[6-oxo-5-(trifluoromethyl)-1-[[2-(trimethylsilyl)ethoxy]methyl]-1,6-dihydropyridazin-4-yl]-2,3-dihydro-1H-isoindol-5-yl]oxy) piperidine-1-carboxylate

A solution of 5-(5-hydroxy-2,3-dihydro-1H-isoindol-2-yl)-4-(trifluoromethyl)-2-[[2-(trimethylsilyl)ethoxy]methyl]-2,3-dihydropyridazin-3-one (4.5 g, 10.53 mmol, 1.00 equiv), tert-butyl 4-iodopiperidine-1-carboxylate (20 g, 64.28 mmol, 8.00 equiv), potassium carbonate (15 g, 108.53 mmol, 10.00 equiv), and DMF (50 mL) was stirred for 2 days at 80° C. The resulting solution was extracted with 2×200 mL of ethyl acetate and the organic layers combined and concentrated under reduced pressure. The residue was applied onto a silica gel column eluting with ethyl acetate/petroleum ether to afford the title compound (2 g, 31%) as a yellow oil. LCMS: [M+H]⁺ 611.15.

### Step 3: 5-[5-(Piperidin-4-yloxy)-2,3-dihydro-1H-isoindol-2-yl]-4-(trifluoromethyl)-2-[[2-(trimethylsilyl)ethoxy]methyl]-2,3-dihydropyridazin-3-one

A solution of tert-butyl 4-([2-[6-oxo-5-(trifluoromethyl)-1-[[2-(trimethylsilyl)ethoxy]methyl]-1,6-dihydropyridazin-4-yl]-2,3-dihydro-1H-isoindol-5 -yl]oxy)piperidine-1-carboxylate (2 g, 3.27 mmol, 1.00 equiv), dioxane/HCl (5 mL), and dioxane (45 mL) was stirred for 6 h at 25° C. The resulting mixture was concentrated under reduced pressure. The residue was applied onto a silica gel column and eluted with ethyl acetate/petroleum ether to afford 1 g of title compound as a yellow oil. LCMS: [M+H]⁺ 511.28.

### Step 4: tert-Butyl 2-[4-([2-[6-oxo-5-(trifluoromethyl)-1-[[2-(trimethylsilyl)ethoxy]methyl]-1,6-dihydropyridazin-4-yl]-2,3-dihydro-1H-isoindol-5-yl]oxy)piperidin-1-yl]acetate

A solution of 5-[5-(piperidin-4-yloxy)-2,3-dihydro-1H-isoindol-2-yl]-4-(trifluoromethyl)-2-[[2-(trimethylsilyl)ethoxy]methyl]-2,3-dihydropyridazin-3-one (1 g, 1.96 mmol, 1.00 equiv), tert-butyl 2-chloroacetate (450 mg, 2.99 mmol, 3.00 equiv), DIPEA (5 mL), and dichloromethane (10 mL) was stirred overnight at 25° C. The residue was purified by C18 reverse phase chromatography eluting with H₂O/CH₃CN to afford the title compound (540 mg, 44%) as a yellow oil. LCMS: [M+H]⁺ 625.20.

### Step 5: 2-[4-([2-[6-Oxo-5-(trifluoromethyl)-1,6-dihydropyridazin-4-yl]-2,3-dihydro-1H-isoindol-5-yl]oxy)piperidin-1-yl]acetic Acid

A solution of tert-butyl 2-[4-([2-[6-oxo-5-(trifluoromethyl)-1-[[2-(trimethylsilyl)ethoxy]methyl]-1,6-dihydropyridazin-4-yl]-2,3-dihydro-1H-isoindol-5-yl]oxy)piperidin-1-yl]acetate (540 mg, 0.86 mmol, 1.00 equiv) and dioxane/HCl (8 mL) was stirred overnight at 25° C. The resulting mixture was concentrated under reduced pressure. The residue was purified by C18 reverse phase chromatography eluting with H₂O/CH₃CN to afford 200 mg (53%) of title compound as a white solid. LCMS: [M+H]⁺ 439.31.

### Example 2: Synthesis of Probe A

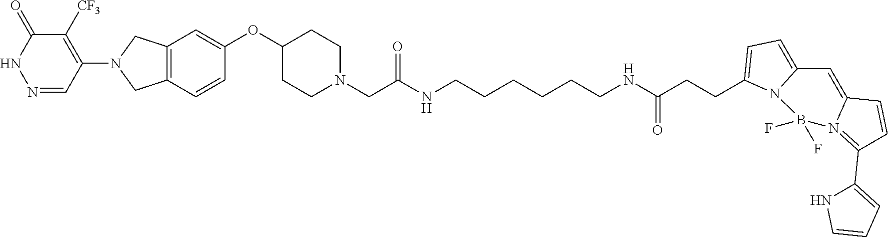

### Step 1: tert-Butyl N-(6-[2-[4-([2-[6-oxo-5-(trifluoromethyl)-1,6-dihydropyridazin-4-yl]-2,3-dihydro-1H-isoindol-5-yl]oxy)piperidin-1-yl]acetamido]hexyl)carbamate

A solution of 2-[4-([2-[6-oxo-5-(trifluoromethyl)-1,6-dihydropyridazin-4-yl]-2,3-dihydro-1H-isoindol-5-yl]oxy)piperidin-1-yl]acetic acid (Int-A2) (44 mg, 0.10 mmol, 1.00 equiv), DIPEA (52 mg, 0.40 mmol, 4.00 equiv), HATU (46 mg, 0.12 mmol, 1.20 equiv), and tert-butyl N-(6-aminohexyl)carbamate (24 mg, 0.11 mmol, 1.10 equiv) in DMF (1 mL) was stirred overnight at 25° C. The crude product was purified by C18 reverse phase chromatography eluting with H₂O/CH₃CN to afford 38 mg (59%) of title compound as an off-white solid. LCMS: [M+H]⁺ 637.31.

### Step 2: N-(6-Aminohexyl)-2-[4-([2-[6-oxo-5-(trifluoromethyl)-1,6-dihydropyridazin-4-yl]-2,3-dihydro-1H-isoindol-5-yl]oxy)piperidin-1-yl]acetamide hydrochloride

A solution of tert-butyl N-(6-[2-[4-([2-[6-oxo-5-(trifluoromethyl)-1,6-dihydropyridazin-4-yl]-2,3-dihydro-1H-isoindol-5-yl]oxy)piperidin-1-yl]acetamido]hexyl)carbamate (38 mg, 0.06 mmol, 1.00 equiv) in hydrogen chloride/dioxane (10 mL) was stirred for 3 hours at 25° C. The resulting mixture was concentrated under reduced pressure to afford the title compound as a gray solid (30 mg, 88%). LCMS: [M−Cl]⁺: 537.27.

### Step 3: 2,2-Difluoro-4-[2-[(6-[2-[4-([2-[6-oxo-5-(trifluoromethyl)-1,6-dihydropyridazin-4-yl]-2,3-dihydro-1H-isoindol-5-yl]oxy)piperidin-1-yl]acetamido]hexyl)carbamoyl]ethyl]-12-(1H-pyrrol-2-yl)-1\hat{ }[5],3-diaza-2\hat{ }[4]-boratricyclo[7.3.0.0\hat{ }[3,7]]dodeca-1(12), 4,6,8,10-pentaen-1-yliumc

A solution of NanoBRET® 590SE (N-(6-aminohexyl)-2-[4-([2-[6-oxo-5-(trifluoromethyl)-1,6-dihydropyridazin-4-yl]-2,3-dihydro-1H-isoindol-5-yl]oxy)piperidin-1-yl]acetamide hydrochloride (23 mg, 0.04 mmol, 2.00 equiv), DIPEA (52 mg, 0.40 mmol, 5.00 equiv), 4-3-[(2,5-dioxopyrrolidin-1-yl)oxy]-3-oxopropyl-2,2-difluoro-12-(1H-pyrrol-2-yl)-1\hat{ }5,3-diaza-2\hat{ }4-boratricyclo[7.3.0.0\hat{ }3,7]dodeca-1(12),4,6,8,10-pentaen-1-ylium) (10 mg, 0.02 mmol, 1.00 equiv) in dichloromethane (2 mL) and methanol (2 mL) was stirred for 2 h at 25° C. The resulting mixture was concentrated under reduced pressure and the crude product was purified by C18 reverse phase chromatography eluting with H₂O/CH₃CN to afford 6.9 mg of a purple solid (35%). LCMS: [M+H]⁺ 848.38.

¹H NMR (CD₃OD, 400 MHz) δ: 7.98 (s, 1H), 7.28-7.14 (m, 5H), 7.02-6.84 (m, 4H), 6.37-6.26 (m, 2H), 4.93 (d, J=12.0 Hz, 4H), 4.45-4.35 (m, 1H), 3.29-3.13 (m, 6H), 3.01 (s, 2H), 2.81-2.70 (m, 2H), 2.60 (t, J=7.7 Hz, 2H), 2.43 (td, J=8.7, 4.6 Hz, 2H), 2.01 (dd, J=11.8, 7.0 Hz, 2H), 1.81 (ddt, J=15.8, 11.5, 5.5 Hz, 2H), 1.51 (q, J=7.3, 6.8 Hz, 4H), 1.38-1.26 (m, 4H).

### Example 3: Synthesis of Probe B

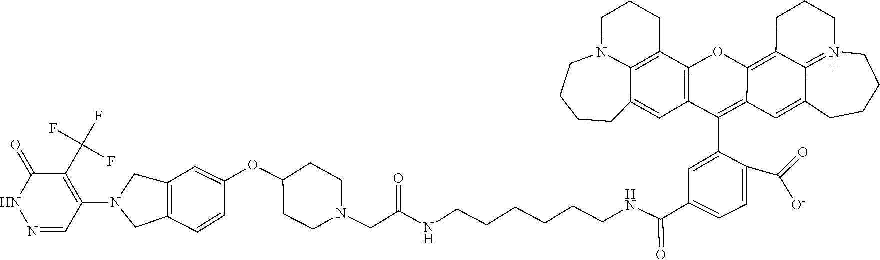

### Step 1: tert-Butyl N-(6-[2-[4-([2-[6-oxo-5-(trifluoromethyl)-1,6-dihydropyridazin-4-yl]-2,3-dihydro-1H-isoindol-5-yl]oxy)piperidin-1-yl]acetamido]hexyl)carbamate

A solution of 2-[4-([2-[6-oxo-5-(trifluoromethyl)-1,6-dihydropyridazin-4-yl]-2,3-dihydro-1H-isoindol-5-yl]oxy)piperidin-1-yl]acetic acid (Int-A2) (250 mg, 0.57 mmol, 1.00 equiv), tert-butyl N-(6-aminohexyl)carbamate (120 mg, 0.55 mmol, 1.00 equiv), HATU (220 mg, 0.58 mmol, 1.10 equiv), DIPEA (2 mL), and DMF (4 mL) was stirred for 0.5 h at 0° C. The residue was purified by C18 reverse phase chromatography eluting with H₂O/CH₃CN to afford 190 mg (52%) of the title compound as a white solid. LCMS: [M+H]⁺ 637.32.

### Step 2: N-(6-Aminohexyl)-2-[4-([2-[6-oxo-5-(trifluoromethyl)-1,6-dihydropyridazin-4-yl]-2,3-dihydro-1H-isoindol-5-yl]oxy)piperidin-1-yl]acetamide hydrochloride

A solution of tert-butyl N-(6-[2-[4-([2-[6-oxo-5-(trifluoromethyl)-1,6-dihydropyridazin-4-yl]-2,3-dihydro-1H-isoindol-5-yl]oxy)piperidin-1-yl]acetamido]hexyl)carbamate (190 mg, 0.30 mmol, 1.00 equiv) and dioxane/HCl (6 mL) was stirred for 1 hour at 25° C. The resulting mixture was concentrated under reduced pressure. The residue was purified by C18 reverse phase chromatography eluting with H₂O/CH₃CN to afford 100 mg (58%) of title compound as a yellow oil. LCMS: [M+H]+ 537.27.

### Step 3: 17-[2-Carboxylato-5-[(6-[2-[4-([2-[6-oxo-5-(trifluoromethyl)-1,6-dihydropyridazin-4-yl]-2,3-dihydro-1H-isoindol-5-yl]oxy)piperidin-1-yl]acetamido]hexyl)carbamoyl]phenyl]-3-oxa-9\hat{ }[5],25-diazaheptacyclo[18.8.1.1\hat{ }[5,9].0\hat{ }[2,18].0\hat{ }[4,16].0\hat{ }[25,29].0\hat{ }[14,30]]triaconta-1(29),2(18),4,9(30),14,16,19-heptaen-9-ylium

A solution of N-(6-aminohexyl)-2-[4-([2-[6-oxo-5-(trifluoromethyl)-1,6-dihydropyridazin-4-yl]-2,3-dihydro-1H-isoindol-5-yl]oxy)piperidin-1-yl]acetamide hydrochloride (8 mg, 0.01 mmol, 1.00 equiv), 17-[2-carboxylato-5-(2,3,5,6-tetrafluorophenoxycarbonyl)phenyl]-3-oxa-9\hat{ }5,25-diazaheptacyclo[18.8.1.1\hat{ }5,9.0\hat{ }2,18.0\hat{ }4,16.0\hat{ }25,29.0\hat{ }14,30]triaconta-1(29),2(18),4,9(30),14,16,19-heptaen-9-ylium (NanoBRET® 618TFP Ester) (10 mg, 0.01 mmol, 1.00 equiv), DIPEA (0.8 mL), and DMF (6 mL) was stirred for 1 h at 25° C. The resulting mixture was concentrated under reduced pressure. The crude product was purified by Prep-HPLC (XBridge Prep C18 OBD column, 5 μm, 19×150 mm column, eluting with water: acetonitrile (50:50, v:v) with 0.1% NH₄HCO₃, at a flow rate of 1.2 mL/min) to afford the title compound as a blue solid (0.8 mg, 5%). LCMS: [M+H]⁺ 1081.45.

¹H NMR (CD₃OD, 400 MHz) δ: 8.09-8.02 (m, 3 H), 7.68 (d, J=1.6 Hz, 1 H), 7.22 (d, J=8.4 Hz, 1 H), 6.93-6.83 (m, 4 H), 4.91 (s, 4 H), 4.55-4.51 (m, 1 H), 3.72-3.69 (m, 3 H), 3.49-3.54 (m, 3 H), 3.35-3.42 (m, 2 H), 3.27-3.23 (m, 2 H), 3.15-3.01 (m, 5 H), 2.84-2.81 (m, 5 H), 2.45-2.41 (m, 2 H), 2.08-1.92 (m, 15 H), 1.84-1.80 (m, 4 H), 1.66-1.37 (m, 7 H), 0.92-0.85 (m, 2 H).

### Example 4: Synthesis of Probe C

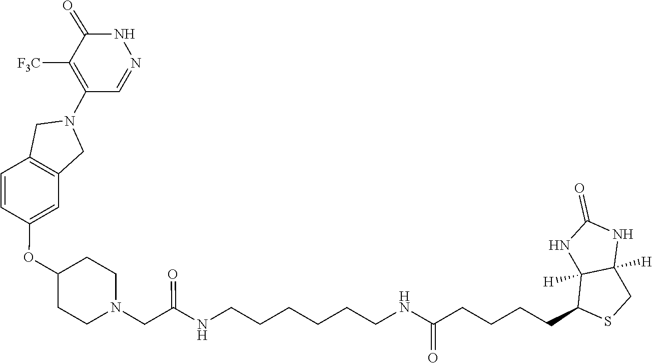 [SMILES: O=C(CCCC[C@@H]1SC[C@@H]2NC(=O)N[C@@H]21)NCCCCCCNC(=O)CN1CCC(Oc2ccc3c(c2)CN(c2cn[nH]c(=O)c2C(F)(F)F)C3)CC1]

### Step 1: Tert-butyl N-(6-[5-[(3aS,4S,6aR)-2-oxo-hexahydro-1H-thieno[3,4-d]imidazolidin-4-yl]pentanamido]hexyl)carbamate

A solution of 5-[(3aS,4S,6aR)-2-oxo-hexahydro-1H-thieno[3,4-d]imidazolidin-4-yl]pentanoic acid ((reagent was purchased from Beijing Dragon Rui Trading Company, 976 mg, 3.99 mmol, 1.00 equiv), DIPEA (1.55 g, 11.99 mmol, 3.00 equiv), HATU (1.82 g, 4.79 mmol, 1.20 equiv), and tert-butyl N-(6-aminohexyl)carbamate (864 mg, 3.99 mmol, 1.00 equiv) in DMF (15 mL) was stirred overnight at 25° C. The reaction was then quenched by the addition of 50 mL of water. The solids were collected by filtration to afford 1.5 g (85%) of the title compound as a white solid. LCMS: [M+H]⁺ 443.26.

### Step 2: 5-[(3aS,4S,6aR)-2-Oxo-hexahydro-1H-thieno[3,4-d]imidazolidin-4-yl]-N-(6-aminohexyl)pentanamide hydrochloride

A solution of tert-butyl N-(6-[5-[(3aS,4S,6aR)-2-oxo-hexahydro-1H-thieno[3,4-d]imidazolidin-4-yl]pentanamido]hexyl)carbamate (800 mg, 1.81 mmol, 1.00 equiv) in hydrogen chloride/dioxane (20 mL) was stirred overnight at 25° C. The resulting mixture was concentrated under reduced pressure to afford 600 mg (88%) of the title compound as a gray crude oil. LCMS: [M+H]⁺ 343.21.

### Step 3: 5-[(3aS,4S,6aR)-2-Oxo-hexahydro-1H-thieno[3,4-d]imidazolidin-4-yl]-N-(6-[2-[4-([2-[6-oxo-5-(trifluoromethyl)-1,6-dihydropyridazin-4-yl]-2, 3-dihydro-1H-isoindol-5-yl]oxy)piperidin-1-yl]acetamido]hexyl)pentanamide

A solution of 2-[4-([2-[6-oxo-5-(trifluoromethyl)-1,6-dihydropyridazin-4-yl]-2,3-dihydro-1H-isoindol-5-yl]oxy)piperidin-1-yl]acetic acid (175 mg, 0.40 mmol, 1.00 equiv), DIPEA (258 mg, 2.00 mmol, 5.00 equiv), HATU (228 mg, 0.60 mmol, 1.50 equiv), 5-[(3aS,4S,6aR)-2-oxo-hexahydro-1H-thieno[3,4-d]imidazolidin-4-yl]-N-(6-aminohexyl)pentanamide hydrochloride (228 mg, 0.60 mmol, 1.50 equiv) in DMF (3 mL) was stirred for 4 h at 25° C. The crude product was purified by C18 reverse phase chromatography eluting with H₂O/CH₃CN to afford the title compound as a white solid (118.3 mg, 39%). LCMS: [M+H]⁺ 763.35.

¹H NMR (DMSO-d₆, 400 MHz) δ: 12.52 (s, 1H), 7.98 (s, 1H), 7.81-7.68 (m, 2H), 7.26 (d, J=8.4 Hz, 1H), 7.00 (d, J=2.2 Hz, 1H), 6.91 (dd, J=8.4, 2.3 Hz, 1H), 6.45-6.39 (m, 1H), 6.36 (s, 1H), 4.91 (d, J=6.1 Hz, 4H), 4.45 (m, 1H), 4.26 (m, 1H), 4.17-4.08 (m, 1H), 3.14-2.96 (m, 5H), 2.91 (s, 2H), 2.82 (dd, J=12.4, 5.1 Hz, 1H), 2.73-2.63 (m, 2H), 2.58 (d, J=12.4 Hz, 1H), 2.33 (ddd, J=11.8, 9.4, 3.1 Hz, 2H), 2.11-1.90 (m, 4H), 1.76-1.54 (m, 3H), 1.57-1.20 (m, 13H).

### Example 5: Synthesis of Probe D

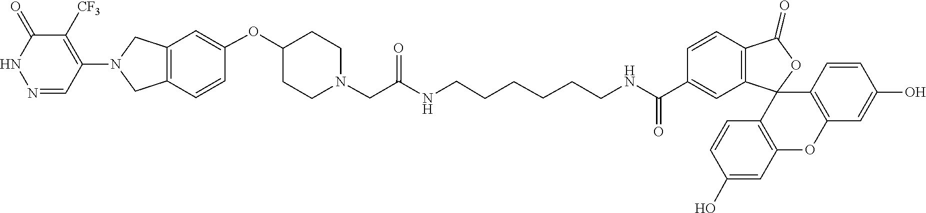 [SMILES: O=C(CN1CCC(Oc2ccc3c(c2)CN(c2cn[nH]c(=O)c2C(F)(F)F)C3)CC1)NCCCCCCNC(=O)c1ccc2c(c1)C1(OC2=O)c2ccc(O)cc2Oc2cc(O)ccc21]

### Step 1: Tert-butyl 2-(4-hydroxypiperidin-1-yl)acetate

A solution of piperidin-4-ol (10.1 g, 99.85 mmol, 1.00 equiv), DIPEA (14.2 g, 109.87 mmol, 1.10 equiv), tert-butyl 2-chloroacetate (14.5 g, 96.28 mmol, 1.00 equiv) in THF (500 mL) was stirred overnight at 25° C. The solids were filtered and concentration under reduced pressure afforded the crude residue which was purified by silica gel chromatography eluting with EtOAC/petroleum ether (1/1) to afford 10.2 g (47%) of the title compound as a white solid. LCMS: [M+H]⁺ 216.15.

### Step 2: Tert-butyl 2-[4-(methanesulfonyloxy)piperidin-1-yl]acetate

A solution of tert-butyl 2-(4-hydroxypiperidin-1-yl)acetate (10.2 g, 47.38 mmol, 1.00 equiv), TEA (9.53 g, 94.18 mmol, 2.00 equiv), Ms₂O (9.86 g, 1.20 equiv) in DCM (200 mL) was stirred for 3 hours at 25° C. The reaction was then quenched by the addition of 300 mL of water. The resulting solution was extracted with 200 mL of dichloromethane and the organic layers were combined. The resulting mixture was concentrated under reduced pressure to afford 11 g (79%) of title compound as an off-white solid. LCMS: [M+H]⁺ 294.13.

### Step 3: Tert-butyl 2-[4-([2-[6-oxo-5-(trifluoromethyl)-1-[[2-(trimethylsilyl)ethoxy]methyl]-1,6-dihydropyridazin-4-yl]-2,3-dihydro-1H-isoindol-5-yl]oxy)piperidin-1-yl]acetate

A solution of 5-(5-hydroxy-2,3-dihydro-1H-isoindol-2-yl)-4-(trifluoromethyl)-2-[[2-(trimethylsilyl)ethoxy]methyl]-2,3-dihydropyridazin-3-one (4.27 g, 9.99 mmol, 1.00 equiv), potassium carbonate (6.9 g, 49.92 mmol, 5.00 equiv), tert-butyl 2-[4-(methanesulfonyloxy)piperidin-1-yl]acetate (5.86 g, 19.97 mmol, 2.00 equiv) in DMF (50 mL) was stirred for 2 days at 80° C. in an oil bath. The reaction was then quenched by the addition of 100 mL of water. The resulting solution was extracted with 4×100 mL of EtOAc and the organic layers were combined. After concentration, the residue was purified by C18 reverse phase chromatography eluting with H₂O/CH₃CN to afford 1.5 g (24%) of the title compound as a light yellow solid. LCMS: [M+H]⁺ 625.30.

### Step 4: 2-[4-([2-[6-Oxo-5-(trifluoromethyl)-1,6-dihydropyridazin-4-yl]-2,3-dihydro-1H-isoindol-5-yl]oxy)piperidin-1-yl]acetic acid

A solution of tert-butyl 2-[4-([2-[6-oxo-5-(trifluoromethyl)-1-[[2-(trimethylsilyl)ethoxy]methyl]-1,6-dihydropyridazin-4-yl]-2,3-dihydro-1H-isoindol-5-yl]oxy)piperidin-1-yl]acetate (1.5 g, 2.40 mmol, 1.00 equiv) in hydrogen chloride/dioxane (40 mL) was stirred overnight at 25° C. The resulting mixture was concentrated under vacuum. The crude product was purified by C18 reverse phase chromatography eluting with H₂O/CH₃CN to afford 600 mg (57%) of the title compound as a gray solid. LCMS: [M+H]⁺ 439.15.

### Step 5: Tert-butyl N-(6-[3′,6′-dihydroxy-3-oxo-3H-spiro[2-benzofuran-1,9′-xanthene]-6-ylformamido]hexyl)carbamate

A solution of 3′,6′-dihydroxy-3-oxo-3H-spiro[2-benzofuran-1,9′-xanthene]-6-carboxylic acid (752 mg, 2.00 mmol, 1.00 equiv), DIPEA (774 mg, 5.99 mmol, 3.00 equiv), HATU (912 mg, 2.40 mmol, 1.20 equiv), tert-butyl N-(6-aminohexyl)carbamate (475 mg, 2.20 mmol, 1.00 equiv) in DMF (10 mL) was stirred for 3 h at 25° C. The reaction was then quenched by the addition of 50 mL of water. The solids were filtered and the crude product was purified by C18 reverse phase chromatography eluting with H₂O/CH₃CN to afford 400 mg (35%) of the title compound as a yellow solid. LCMS: [M+H]⁺ 575.23.

### Step 6: N-(6-Aminohexyl)-3′,6′-dihydroxy-3-oxo-3H-spiro[2-benzofuran-1,9′-xanthene]-6-carboxamide

A solution of tert-butyl N-(6-[3′,6′-dihydroxy-3-oxo-3H-spiro[2-benzofuran-1,9′-xanthene]-6-ylformamido]hexyl)carbamate (400 mg, 0.70 mmol, 1.00 equiv) in HCl/dioxane (20 mL) was stirred overnight at 25° C. The resulting mixture was concentrated under reduced pressure and the crude product was purified by C18 reverse phase chromatography eluting with H₂O/CH₃CN to afford 200 mg of the title compound (61%) as a yellow solid. LCMS: [M+H]⁺ 475.18.

### Step 7: N-(6-[3-Dihydroxy-3-oxo-3H-spiro[2-benzofuran-1,9′-xanthene]]-6-ylformamido]hexyl)-2-[4-([2-[6-oxo-5-(trifluoromethyl)-1,6-dihydropyridazin-4-yl]-2, 3-dihydro-1H-isoindol-5-yl]oxy)piperidin-1-yl]acetamide

A solution of 2-[4-([2-[6-oxo-5-(trifluoromethyl)-1,6-dihydropyridazin-4-yl]-2,3-dihydro-1H-isoindol-5-yl]oxy)piperidin-1-yl]acetic acid (Int-A2) (150 mg, 0.34 mmol, 1.00 equiv), DIPEA (176 mg, 1.36 mmol, 4.00 equiv), HATU (183 mg, 0.48 mmol, 1.40 equiv), N-(6-aminohexyl)-3-dihydroxy-3-oxo-3H-spiro[2-benzofuran-1,9′-xanthene]-6-carboxamide (228 mg, 0.48 mmol, 1.40 equiv) in DMF (5 mL) was stirred for 3 h at 25° C. After concentration, the residue was purified by C18 reverse phase chromatography eluting with H₂O/CH₃CN to afford the title compound as an orange solid (26.9 mg, 9%). LCMS: [M+H]⁺ 895.32.

¹H NMR (DMSO-d₆, 400 MHz) δ: 8.61 (t, J=5.6 Hz, 1H), 8.14-8.02 (m, 2H), 7.96 (s, 1H), 7.72-7.61 (m, 2H), 7.22 (d, J=8.4 Hz, 1H), 6.96 (d, J=2.2 Hz, 1H), 6.86 (dd, J=8.4, 2.2 Hz, 1H), 6.58 (d, J=8.8 Hz, 4H), 6.49 (s, 2H), 4.88 (d, J=5.4 Hz, 4H), 4.34 (dp, J=8.2, 3.7 Hz, 1H), 3.17 (q, J=6.2 Hz, 2H), 3.03 (q, J=6.6 Hz, 2H), 2.86 (s, 2H), 2.70 -2.58 (m, 2H), 2.33-2.22 (m, 2H), 1.91 (d, J=13.5 Hz, 2H), 1.65 (dtd, J=12.6, 8.8, 3.2 Hz, 2H), 1.39 (dq, J=27.4, 6.8, 6.2 Hz, 4H), 1.22 (d, J=6.8 Hz, 4H).

### Example 6: Synthesis of Compound A

### 5-[5-(Piperidin-4-yloxy)-2,3-dihydro-1H-isoindol-2-yl]-4-(trifluoromethyl)-2,3-dihydropyridazin-3-one

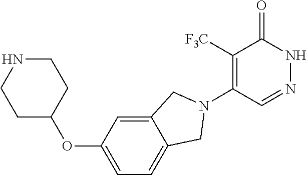 [SMILES: O=c1[nH]ncc(N2Cc3ccc(OC4CCNCC4)cc3C2)c1C(F)(F)F]

### Step 1: 5-(5-Hydroxy-2,3-dihydro-1H-isoindol-2-yl)-4-(trifluoromethyl)-2-[[2-(trimethylsilyl)ethoxy]methyl]-2,3-dihydropyridazin-3-one

A solution of Int-A1 (2.8 g, 8.52 mmol, 1.00 equiv), 2,3-dihydro-1H-isoindol-5-ol hydrobromide (4.27 g, 19.76 mmol, 1.00 equiv), and TEA (10 mL) in ethanol (40 mL) was stirred for 1 h at 60° C. The resulting solution was extracted with 2×100 mL of EtOAc and the organic layers combined and concentrated under reduced pressure to afford 4.5 g of the title compound as a yellow oil. LCMS: [M+H]⁺ 428.23.

### Step 2: tert-Butyl 4-([2-[6-oxo-5-(trifluoromethyl)-1-[[2-(trimethylsilyl)ethoxy]methyl]-1,6-dihydropyridazin-4-yl]-2,3-dihydro-1H-isoindol-5-yl]oxy)piperidine-1-carboxylate

A solution of 5-(5-hydroxy-2,3-dihydro-1H-isoindol-2-yl)-4-(trifluoromethyl)-2-[[2-(trimethylsilyl)ethoxy]methyl]-2,3-dihydropyridazin-3-one (4.5 g, 10.53 mmol, 1.00 equiv), tert-butyl 4-iodopiperidine-1-carboxylate (20 g, 64.28 mmol, 8.00 equiv), potassium carbonate (15 g, 108.53 mmol, 10.00 equiv), and DMF (50 mL) was stirred for 2 days at 80° C. The resulting solution was extracted with 2×200 mL of EtOAc and the organic layers combined and concentrated under reduced pressure. The residue was applied onto a silica gel column eluting with EtOAc/petroleum ether to afford the title compound (2 g, 31%) as a yellow oil. LCMS: [M+H]⁺ 611.15.

### Step 3: 5-[5-(Piperidin-4-yloxy)-2,3-dihydro-1H-isoindol-2-yl]-4-(trifluoromethyl)-2,3-dihydropyridazin-3-one

A solution of tert-butyl 4-([2-[6-oxo-5-(trifluoromethyl)-1-[[2-(trimethylsilyl)ethoxy]methyl]-1,6-dihydropyridazin-4-yl]-2,3-dihydro-1H-isoindol-5-yl]oxy)piperidine-1-carboxylate (150 mg, 0.25 mmol, 1.00 equiv) in HCl/dioxane (5 mL) was stirred overnight at 45° C. The resulting mixture was concentrated under reduced pressure and the crude product was purified by C18 reverse phase chromatography eluting with H₂O/ACN to afford the title compound as a white solid LCMS: [M+H]⁺ 381.28. ¹H NMR (400 MHz, Methanol-d₄) ϵ 8.05 (s, 1H), 7.27 (d, J=8.4 Hz, 1H), 7.02-6.91 (m, 2H), 5.00 (d, J=10.6 Hz, 4H), 4.61-4.48 (m, 1H), 3.21-3.10 (m, 2H), 2.89-2.78 (m, 2H), 2.11-2.08 (m, 2H), 1.82-1.69 (m, 2H).

### Example 7: Synthesis of Compound B

### 6-[4-[(3-[[(1S)-2-[6-Oxo-5-(trifluoromethyl)-1,6-dihydropyridazin-4-yl]-2,3-dihydro-1H-isoindol-1-yl]methoxy]phenyl)carbonyl]piperazin-1-yl]pyridine-3-carbonitrile

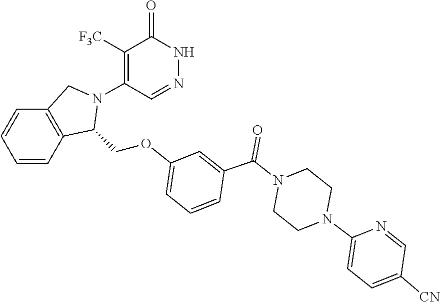 [SMILES: N#Cc1ccc(N2CCN(C(=O)c3cccc(OC[C@@H]4c5ccccc5CN4c4cn[nH]c(=O)c4C(F)(F)F)c3)CC2)nc1]

### Step 1: 5-[1-(Hydroxymethyl)-2,3-dihydro-1H-isoindol-2-yl]-4-(trifluoromethyl)-2-[[2-(trimethylsilyl)ethoxy]methyl]-2,3-dihydropyridazin-3-one

A solution of 5-chloro-4-(trifluoromethyl)-2-[2-(trimethylsilyl)ethoxy]methyl-2,3-dihydropyridazin-3-one (4.8 g, 14.60 mmol, 1.00 equiv), 2,3-dihydro-1H-isoindol-1-ylmethanol hydrochloride (2.7 g, 14.54 mmol, 1.00 equiv) and TEA (4.4 g, 43.48 mmol, 2.99 equiv) in ethanol (100 mL) was stirred for 1 h at 60° C., and then the resulting solution was concentrated under vacuum and the residue was applied onto a silica gel column eluting with EtOAc/petroleum ether (45:55) to afford 2.9 g (45%) of the title compound as a brown solid. LCMS: [M+H]⁺ 442.17.

### Step 2: Methyl 3-([2-[6-oxo-5-(trifluoromethyl)-1-[[2-(trimethylsilyl)ethoxy]methyl]-1,6-dihydropyridazin-4-yl]-2,3-dihydro-1H-isoindol-1-yl]methoxy)benzoate

Under nitrogen, a solution of 5-[1-(hydroxymethyl)-2,3-dihydro-1H-isoindol-2-yl]-4-(trifluoromethyl)-2-[[2-(trimethylsilyl)ethoxy]methyl]-2,3-dihydropyridazin-3-one (2.93 g, 6.64 mmol, 1.00 equiv), methyl 3-bromobenzoate (2.84 g, 13.21 mmol, 1.99 equiv), Pd(allyl)Cl₂ (243 mg), Rockphos (311 mg) and Cs₂CO₃ (4.3 g, 13.20 mmol, 1.99 equiv) in Toluene (100 mL) was stirred for 18 h at 80° C. The resulting solution was concentrated under vacuum and then the residue was applied onto a silica gel column eluting with EtOAc/petroleum ether (1:3) to afford 3 g (79%) of the title compound as a brown solid. LCMS: [M+H]⁺ 576.21.

### Step 3: 3-([2-[6-Oxo-5-(trifluoromethyl)-1-[[2-(trimethylsilyl)ethoxy]methyl]-1,6-dihydropyridazin-4-yl]-2,3-dihydro-1H-isoindol-1-yl]methoxy)benzoic acid

A solution of methyl 3-([2-[6-oxo-5-(trifluoromethyl)-1-[[2-(trimethylsilyl)ethoxy]methyl]-1,6-dihydropyridazin-4-yl]-2,3-dihydro-1H-isoindol-1-yl]methoxy)benzoate (1.15 g, 2.00 mmol, 1.00 equiv) and LiOH (240 mg, 10.02 mmol, 5.02 equiv) in THF (12 mL) and water (3 mL) was stirred for 3 h at 60° C. The resulting solution was concentrated under vacuum and the residue was diluted with 10 mL of H₂O, and then the pH value of the solution was adjusted to 5 with HCl (36.5%). The solid was collected by filtration to afford 1.1 g (98%) of the title compound as a light yellow solid. LCMS: [M+H]⁺ 562.19.

### Step 4: 3-([2-[6-Oxo-5-(trifluoromethyl)-1,6-dihydropyridazin-4-yl]-2,3-dihydro-1H-isoindol-1-yl]methoxy)benzoic acid

A solution of 3-([2-[6-oxo-5-(trifluoromethyl)-1-[[2-(trimethylsilyl)ethoxy]methyl]-1,6-dihydropyridazin-4-yl]-2,3-dihydro-1H-isoindol-1-yl]methoxy)benzoic acid (1.1 g, 1.96 mmol, 1.00 equiv) in HCl/dioxane (20 mL, 4M) was stirred for 3 h at RT, and then the resulting solution was concentrated under vacuum to afford 1 g of the title compound as a crude brown solid. LCMS: [M+H]⁺ 432.11.

### Step 5: 6-[4-[(3-[[(1R)-2-[6-oxo-5-(trifluoromethyl)-1,6-dihydropyridazin-4-yl]-2,3-dihydro-1H-isoindol-1-yl]methoxy]phenyl)carbonyl]piperazin-1-yl]pyridine-3-carbonitrile and 6-[4-[(3-[[(1S)-2-[6-oxo-5-(trifluoromethyl)-1,6-dihydropyridazin-4-yl]-2,3-dihydro-1H-isoindol-1-yl]methoxy]phenyl)carbonyl]piperazin-1-yl]pyridine-3-carbonitrile

A solution of 3-([2-[6-oxo-5-(trifluoromethyl)-1,6-dihydropyridazin-4-yl]-2,3-dihydro-1H-isoindol-1-yl]methoxy)benzoic acid (500 mg, 1.16 mmol, 1.00 equiv), HATU (528 mg, 1.39 mmol, 1.20 equiv), DIPEA (449 mg, 3.47 mmol, 3.00 equiv) and Int-A4 (240 mg, 1.27 mmol, 1. lequiv) in DMF (5 mL) was stirred for 2 h at RT. After concentration by reduced pressure, the resulting solution was purified by C18 reverse phase chromatography eluting with H₂O/ACN. The residue was further purified by Prep-HPLC and Chiral-Prep-HPLC (CHIRAL Repaired IA, 5 μm, 0.46×10 cm column, eluting with a gradient of (Hexanes: DCM=3:1) (0.1% DEA): EtOH=50:50, at a flow rate of 1 mL/min) yielding the title compound as a white solid. The absolute stereochemistry was assigned based on an X-ray crystal structure which confirmed(S)-absolute stereochemistry.

LCMS: [M+H]⁺ 602.05, ¹H NMR (300 MHz, Methanol-d₄) δ 8.43 (d, J=1.8 Hz, 1H), 8.42 (s, 1H), 7.79 (dd, J=9.0, 2.4 Hz, 1H), 7.53-7.50 (m, 1H), 7.41-7.35 (m, 4H), 7.05-6.99 (m, 2H), 6.94-6.87 (m, 2H), 6.20 (s, 1H), 5.33 (d, J=14.8 Hz, 1H), 4.68 (d, J=14.7 Hz, 1H), 4.53 (dd, J=10.2, 3.3 Hz, 1H), 4.29 (dd, J=10.2, 6.6 Hz, 1H), 3.91-3.44 (m, 8H). tR =5.955 min.

### Example 8: Synthesis of Compound C

### 5-[2-[(1-Acetylpiperidin-4-yl)oxy]-5H,6H,7H-pyrrolo[3,4-b]pyridin-6-yl]-4-(trifluoromethyl)-2,3-dihydropyridazin-3-one

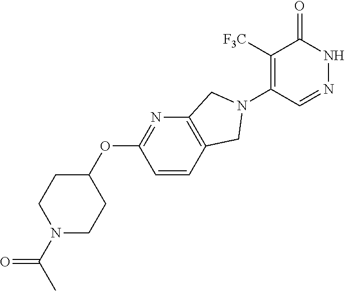 [SMILES: CC(=O)N1CCC(Oc2ccc3c(n2)CN(c2cn[nH]c(=O)c2C(F)(F)F)C3)CC1]

### Step 1: 5-[2-Chloro-5H,6H,7H-pyrrolo[3, 4-b]pyridin-6-yl]-4-(trifluoromethyl)-2-[[2-(trimethylsilyl)ethoxy]methyl]-2,3-dihydropyridazin-3-one

A solution of 2-chloro-5H,6H,7H-pyrrolo[3,4-b]pyridin-6-yl hydrochloride (5 g, 26.31 mmol, 1.00 equiv), TEA (8 g, 79.06 mmol, 3.00 equiv), and Int-A1 (14.3 g, 43.49 mmol, 1.00 equiv) in EtOH (30 mL) was stirred for 2 h at 80° C. After concentration under reduced pressure, the residue was applied onto a silica gel column with EtO Ac/petroleum ether (1:4) to afford 9.3 g (79%) of title compound as a yellow oil. LCMS: [M+H]⁺ 447.15.

### Step 2: 5-[2-Hydroxy-5H,6H,7H-pyrrolo[3,4-b]pyridin-6-yl]-4-(trifluoromethyl)-2-[[2-(trimethylsilyl)ethoxy]methyl]-2,3-dihydropyridazin-3-one

A solution of 5-[2-chloro-5H,6H,7H-pyrrolo[3,4-b]pyridin-6-yl]-4-(trifluoromethyl)-2-[[2-(trimethylsilyl)ethoxy]methyl]-2,3-dihydropyridazin-3-one (300 mg, 0.67 mmol, 1.00 equiv), tBuBrettphos (49 mg, 0.15 equiv), K₃PO₄ (427 mg, 2.01 mmol, 3.00 equiv), and Pd(OAc)₂ (15 mg, 0.07 mmol, 0.10 equiv) in dioxane (5 mL) and water (0.5 mL) was stirred for 2 h at 80° C. in an oil bath under N₂ atmosphere. After concentration, the residue was applied onto a silica gel column with DCM/methanol (85:15) to afford 200 mg (70%) of title compound as a yellow oil. LCMS: [M+H]⁺ 429.15

### Step 3: Tert-butyl 4-([6-[6-oxo-5-(trifluoromethyl)-1-[[2-(trimethylsilyl)ethoxy]methyl]-1,6-dihydropyridazin-4-yl]-5H,6H,7H-pyrrolo[3,4-b]pyridin-2-yl]oxy)piperidine-1-carboxylate

A solution of 5-[2-hydroxy-5H,6H,7H-pyrrolo[3,4-b]pyridin-6-yl]-4-(trifluoromethyl)-2-[[2-(trimethylsilyl)ethoxy]methyl]-2,3-dihydropyridazin-3-one (200 mg, 0.47 mmol, 1.00 equiv), Ag₂CO₃ (247 mg, 2.00 equiv), and tert-butyl 4-iodopiperidine-1-carboxylate (416 mg, 1.34 mmol, 3.00 equiv) in DMF (15 mL) was stirred for 4 h at 80° C. The resulting solution was extracted with 3×10 mL of EtOAc and the organic layers combined. After concentration, the residue was applied onto a silica gel column with EtOAc/petroleum ether (1:9) to afford 150 mg (53%) of title compound as a yellow oil. LCMS: [M+H]⁺ 612.30.

### Step 4: 5-[2-(Piperidin-4-yloxy)-5H,6H,7H-pyrrolo[3, 4-b]pyridin-6-yl]-4-(trifluoromethyl)-2,3-dihydropyridazin-3-one

A solution of tert-butyl 4-([6-[6-oxo-5-(trifluoromethyl)-1-[[2-(trimethylsilyl)ethoxy]methyl]-1,6-dihydropyridazin-4-yl]-5H,6H,7H-pyrrolo[3,4-b]pyridin-2-yl]oxy)piperidine-1-carboxylate (150 mg, 0.25 mmol, 1.00 equiv) in HCl/dioxane (15 mL, 4M) was stirred overnight at 25° C. The pH value of the solution was adjusted to 8 with ammonia (100%). The crude product was purified by Prep-HPLC to afford 64.8 mg (69%) of title compound as a white solid. LCMS: [M+H]⁺ 382.15 [M+H].

### Step 5: 5-[2-[(1-Acetylpiperidin-4-yl)oxy]-5H,6H,7H-pyrrolo[3,4-b]pyridin-6-yl]-4-(trifluoromethyl)-2,3-dihydropyridazin-3-one

A solution of 5-[2-(piperidin-4-yloxy)-5H,6H,7H-pyrrolo[3,4-b]pyridin-6-yl]-4-(trifluoromethyl)-2,3-dihydropyridazin-3-one (300 mg, 0.79 mmol, 1.00 equiv), TEA (239 mg, 2.36 mmol, 3.00 equiv), and EtOAc (160 mg, 1.57 mmol, 2.00 equiv) in DCM (15 mL) was stirred for 1 h at 25° C. The resulting solution was quenched by 20 mL of water and extracted with 3×15 mL of DCM and the organic layers combined. After concentration, the crude product was purified by Flash-Prep-HPLC to afford 70.2 mg (21%) of title compound as a white solid. LCMS: [M+H]⁺ 424.15 ¹H NMR (400 MHz, Methanol-d4) 8 8.07 (s, 1H), 7.69 (d, J=8.4 Hz, 1H), 6.76 (d, J=8.4 Hz, 1H), 5.39-5.28 (m, 1H), 5.03 (s, 2H), 4.92 (s, 2H), 3.97-3.89 (m, 1H), 3.86-3.75 (m, 1H), 3.57-3.44 (m, 2H), 2.15 (s, 3H), 2.14-1.97 (m, 2H), 1.89-1.70 (m, 2H).

### Example 9: In Vitro Assays

### Recombinant PARP Enzymes

A portion of TIPARP (residues 456 to 657 of NP_056323.2 (SEQ ID NO: 1), GenBank Accession No. NM_015508.4) was overexpressed in E. coli cells. An N-terminal fusion tag, MHHHHHHSSGVDLGTENLYFQSNAGLNDIFEAQKIEWHE (SEQ ID NO: 7), was used to purify the protein from cell lysates. The fusion tag was left on the protein for use in the probe displacement assay.

A portion of PARP10 (residues 808 to 1025 of NP_116178.2 (SEQ ID NO: 2), GenBank Accession No. NM_032789.4) was overexpressed in E. coli cells. An N-terminal fusion tag, MAHHHHHHENLYFQSM (SEQ ID NO: 8), was used to purify the protein from cell lysates. The fusion tag was left on the protein for use in the probe displacement assay.

A portion of PARP12 (residues 489 to 684 of NP_073587.1 (SEQ ID NO: 3), GenBank Accession No. NM_022750.3) was overexpressed in Sf9 cells. An N-terminal fusion tag, MAHHHHHHENLYFQSM (SEQ ID NO: 8), was used to purify the protein from cell lysates. The fusion tag was left on the protein for use in the probe displacement assay.

A portion of PARP14 (residues 1611 to 1801 of NP_060024.2 (SEQ ID NO: 4), GenBank Accession No. NM_017554) was overexpressed in E. coli cells. An N-terminal fusion tag, MHHHHHHSSGVDLGTENLYFQSNA (SEQ ID NO: 9), was used to purify the protein from cell lysates. The fusion tag was left on the protein for use in the probe displacement assay.

A portion of PARP15 (residues 481 to 678 of NP_689828.1 (SEQ ID NO: 5), GenBank Accession No. NM_152615) was overexpressed in Sf9 cells. An N-terminal fusion tag, MAHHHHHHSSGVDLGTENLYFQSM (SEQ ID NO: 10), was used to purify the protein from cell lysates. The fusion tag was left on the protein for use in the probe displacement assay.

A portion of PARP16 (residues 5 to 279 of NP_060321.3 (SEQ ID NO: 6), GenBank Accession No. NM_017851) was overexpressed in E. coli cells. An N-terminal fusion tag, MHHHHHHSSGVDLGTENLYFQSNA (SEQ ID NO: 9), was used to purify the protein from cell lysates. The fusion tag was left on the protein for use in the probe displacement assay.

### In Vitro Probe Displacement Assay for Assessing Binding of Inhibitors to TIPARP, PARP10, PARP 14 and PARP16

Displacement of a Probe C binding to monoPARP active sites was measured using a time-resolved fluorescence resonance energy transfer (TR-FRET) assay. 20 nL of a dose response curve of each test compound was spotted in black 384-well polystyrene proxiplates (Perkin Elmer) using a Mosquito (TTP Labtech). Reactions were performed in an 8 μL volume by adding 6 μL of the monoPARP and Probe C in assay buffer (20 mM HEPES pH=8, 100 mM NaCl, 0.1% bovine serum albumin, 2 mM DTT and 0.002% Tween20), incubating with test compound at 25° C. for 30 min, then adding 2 HL of ULight-anti 6xHis and LANCE Eu-W1024 labeled streptavidin (Perkin Elmer) The final concentrations of monoPARP, Probe C, ULight-anti 6xHis and LANCE Eu-W1024 labeled streptavidin are listed in Table 3. Binding reactions were equilibrated at 25° C. for an additional 30 min, then read on an Envision platereader equipped with a LANCE/DELFIA top mirror (Perkin Elmer) using excitation of 320 nm and emission of 615 nm and 665 nM with a 90 μs delay. The ratio of the 665/615 nm emission were calculated for each well to determine the amount of complex of monoPARP and Probe C in each well.

TABLE 3
Assay conditions for monoPARP probe displacement assays where the
streptavidin is labeled with TR-FRET donor

| | | | | LANCE Eu- |
|---|---|---|---|---|
| | Enzyme | Probe C | ULight-anti | W1024 labeled |
| Target | (nM) | (nM) | 6xHis (nM) | streptavidin (nM) |
| TIPARP | 6 | 2 | 4 | 0.25 |
| PARP10 | 6 | 0.5 | 2 | 0.25 |
| PARP14 | 6 | 2 | 10 | 0.25 |
| PARP16 | 3 | 1 | 6 | 0.25 |

### In Vitro Probe Displacement Assay for Assessing Binding of Inhibitors to PARP12 and PARP15

Displacement of a Probe C binding to monoPARP active sites was measured using a time-resolved fluorescence resonance energy transfer (TR-FRET) assay. 20 nL of a dose response curve of each test compound was spotted in black 384-well polystyrene proxiplates (Perkin Elmer) using a Mosquito (TTP Labtech). Reactions were performed in a 8 μL volume by adding 6 μL of the monoPARP and Probe C in assay buffer (20 mM HEPES pH=8, 100 mM NaCl, 0.1% bovine serum albumin, 2 mM DTT and 0.002% Tween20), incubating with test compound at 25° C. for 30 min, then adding 2 μL. of ULight-streptavidin and LANCE Eu-W1024 Anti-6xHis (Perkin Elmer). The final concentrations of monoPARP, Probe C, ULight-streptavidin and LANCE Eu-W1024 Anti-6xHis are listed in Table 4. Binding reactions were equilibrated at 25° C. for an additional 30 min, then read on an Envision platereader equipped with a LANCE/DELFIA top mirror (Perkin Elmer) using excitation of 320 nm and emission of 615 nm and 665 nM with a 90 μs delay. The ratio of the 665/615 nm emission were calculated for each well to determine the amount of complex of monoPARP and Probe C in each well.

TABLE 4
Assay conditions for monoPARP probe displacement assays where the
anti-His antibody is labeled with TR-FRET donor

| | | | | LANCE Eu- |
|---|---|---|---|---|
| | Enzyme | Probe C | ULight- labeled | W1024 anti |
| Target | (nM) | (nM) | streptavidin (nM) | 6xHis (nM) |
| PARP12 | 6 | 32 | 10 | 1.25 |
| PARP15 | 1.5 | 8 | 2 | 0.5 |

### Data Analysis for All In Vitro Assays

Control wells containing a negative control of 0.25% DMSO vehicle or a positive control of 100 μM Compound A were used to calculate the % inhibition as described below:

$$\% inhibition = 100 \times \frac{{TRF}_{cmpd} - {TRF}_{min}}{{TRF}_{mαx} - {TRF}_{min}}$$

where TRF_{cmpd} is the TR-FRET ratio from the compound treated well, TRFₘᵢₙ is the TR-FRET ratio from the Compound A-treated positive control well and TREₘₐₓ is the TR-FRET ratio from the DMSO-treated negative control well.

The % inhibition values were plotted as a function of compound concentration and the following 4-parameter fit was applied to derive the IC₅₀ values:

$$Y = Bottom + \frac{( Top - Bottom )}{( 1 + {( \frac{X}{{IC}_{50}} )}^{Hill Coefficient}}$$

where top and bottom are normally allowed to float, but may be fixed at 100 or 0 respectively in a 3-parameter fit. The Hill Coefficient is normally allowed to float but may also be fixed at 1 in a 3-parameter fit. Y is the % inhibition and X is the compound concentration.

### Validation of In Vitro Probe Displacement Assays

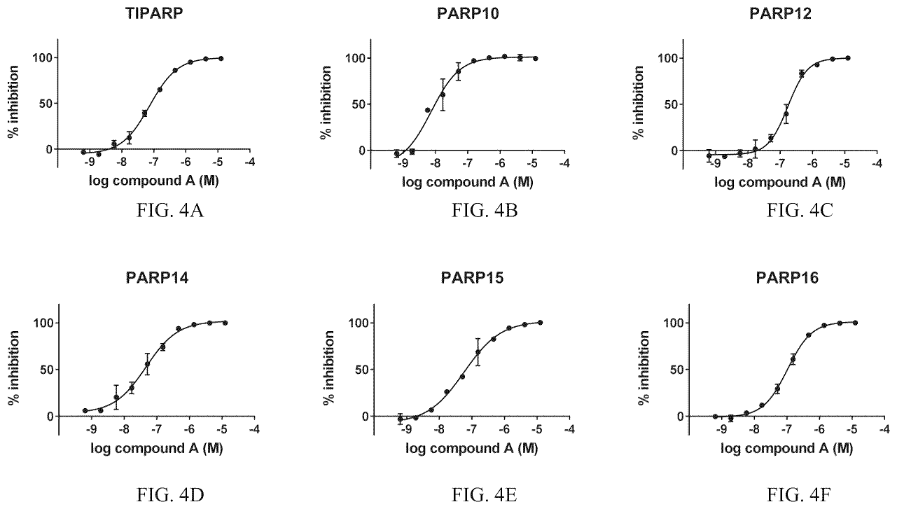

The probe displacement assays were validated by outcompeting Probe C with Compound A, an analog that does not contain a linker or biotin group. The assays for TIPARP, PARP10, PARP12, PARP14, PARP15 and PARP16 were set up as described above, and the results are shown in FIGS. 4A-4F

FIGS. 4A-4F are validation results of the in vitro probe displacement binding assays. Dose response curves for Compound A were generated using each assay to confirm that Probe C was able to be outcompeted from the monoPARP enzyme. IC₅₀ values were TIPARP=7 nM, PARP10=80 nM, PARP12=200 nM, PARP14=50 nM, PARP15=60 nM and PARP16=100 nM.

### Example 10: Live Cell Assays

### NanoLuc Plasmids

MonoPARP genes from Table 5 were cloned into the pcDNA3.1-mammalian expression vector as a NanoLuc fusion with NanoLuc on the N- or C-terminus as indicated. The sequence of the NanoLuc tag is as follows:

NanoLuc amino acid sequence (SEQ ID NO: 11):
MVFTLEDFVGDWRQTAGYNLDQVLEQGGVSSLFQNLGVSVTPIQRIVLSG
ENGLKIDIHVIIPYEGLSGDQMGQIEKIFKVVYPVDDHHFKVILHYGTLV
IDGVTPNMIDYFGRPYEGIAVFDGKKITVTGTLWNGNKIIDERLINPDGS
LLFRVTINGVTGWRLCERILA
NanoLuc nucleic acid sequence (SEQ ID NO: 12):
atggtcttcacactcgaagatttcgttggggactggcgacagacagccgg
ctacaacctggaccaagtccttgaacagggaggtgtgtccagtttgtttc
agaatctcggggtgtccgtaactccgatccaaaggattgtcctgagcggt
gaaaatgggctgaagatcgacatccatgtcatcatcccgtatgaaggtct
gagcggcgaccaaatgggccagatcgaaaaaatttttaaggtggtgtacc
ctgtggatgatcatcactttaaggtgatcctgcactatggcacactggta
atcgacggggttacgccgaacatgatcgactatttcggacggccgtatga
aggcatcgccgtgttcgacggcaaaaagatcactgtaacagggaccctgt
ggaacggcaacaaaattatcgacgagcgcctgatcaaccccgacggctcc
ctgctgttccgagtaaccatcaacggagtgaccggctggcggctgtgcga
acgcattctggcgtaa

TABLE 5
Details for the monoPARP-NanoLuc fusion genes used

| | Genbank | | |
|---|---|---|---|
| | Accession | Amino Acid | NanoLuc |
| Plasmid Name | Number | Residues | Position |
| Full-length TIPARP | NM_015508 | 1-657 of | C-terminus |
| | | NP_056323.2 | |
| | | (SEQ ID NO: 1) | |
| Catalytic domain | NM_015508 | 456-657 of | C-terminus |
| TIPARP | | NP_056323.2 | |
| | | (SEQ ID NO: 1) | |
| PARP10 | NM_032789 | 808-1025 of | C-terminus |
| | | NP_116178.2 | |
| | | (SEQ ID NO: 2) | |
| PARP12 | NM_022750.3 | 489-684 of | C-terminus |
| | | NP_073587.1 | |
| | | (SEQ ID NO: 3) | |
| PARP14 | NM_017554 | 1611-1801 of | N-terminus |
| | | NP_060024.2 | |
| | | (SEQ ID NO: 4) | |

### NanoBRET Probe Displacement Assay for Assessing Binding of Inhibitors to TIP ARP, PARP10, PARP12 and PARP14

Displacement of a fluorescently-labeled compound Probe A binding to NanoLuc-tagged monoPARP enzymes was measured in live cells using a bioluminescence resonance energy transfer (NanoBRET) assay. TIPARP, PARP10, PARP12 or PARP14 fused to a NanoLuc tag were overexpressed in 293T cells (ATCC) using the plasmids described herein. Plasmid DNA and empty vector DNA were added to phenol red free OptiMEM (Thermo Fisher) as shown in Table 6 in a total volume of 2.456 mL. 157 μL of Fugene HD (Promega) was added to the DNA mixture and allowed to incubate 5 min at 25° C.

TABLE 6

| | Concentration of | Concentration of | |
|---|---|---|---|
| | Plasmid | Empty Vector | Probe A |
| Plasmid Name | (μg/mL) | (μg/mL) | (nM) |
| Full-length TIPARP | 0.2 | 19.8 | 9 |
| Catalytic domain | 0.2 | 19.8 | 80 |
| TIPARP | | | |
| PARP10 | 2 | 18 | 7 |
| PARP12 | 2 | 18 | 300 |
| PARP14 | 0.2 | 19.8 | 100 |

Next, 2.375 mL of the plasmid-Fugene mixture were added to 20 million 293T cells in DMEM (Thermo Fisher) supplemented with 10% FBS (VWR). The transfection was incubated for 24 h at 37° C. in an incubator containing air supplemented with 5% CO₂. The cells were resuspended in phenol red free OptiMEM media. Transfected 293T cells were diluted to 500,000 cells per mL and Probe A was added to a final concentration as shown in Table 6. 40 μL of cells were then added to white polystyrene 384-well non-binding surface microplate (Corning). 40 nL of a dose response curve diluted in DMSO of each test compound was added to the cell plate using a Mosquito (TTP Labtech) and the plate was incubated at 37° C. in an incubator containing air supplemented with 5% CO₂ for 2 h. The assay plate was allowed to equilibrate to room temperature (25° C.), then 20 μL per well of NanoBRET substrate (Promega) was added to the plate (1:166 dilution of NanoBRET substrate, 1:500 dilution of NanoLuc extracellular inhibitor in OptiMEM without phenol red). Filtered luminescence was measured on an Envision (Perkin Elmer) equipped with a dual 585 nm mirror, 460±40 nm bandpass filter (donor) and 610±50 nm longpass filter (acceptor).

TABLE 7

| | Positive control | Final Concentration |
|---|---|---|
| Assay | compound | (μM) |
| Full-length TIPARP | Compound B | 0.1 |
| Catalytic domain TIPARP | Compound B | 0.2 |
| PARP10 | Compound C | 2 |
| PARP12 | Compound B | 1 |
| PARP14 | Compound D | 5 |

### Data Analysis for NanoBRET Assays

BRET ratio was measured as shown below:

$$BRET ratio = \frac{Emission at 610 nm}{Luminescence}$$

Control wells containing a negative control of 0.2% DMSO vehicle or a positive control were used to calculate the % inhibition as described below:

$$\% inhibition = 100 \times \frac{BRET {ratio}_{cmpd} - BRET {ratio}_{min}}{BRET {ratio}_{max} - BRET {ratio}_{min}}$$

where BRET ratio_{cmpd} is the BRET ratio from the compound treated well, BRET ratioₘᵢₙ is the BRET ratio from the positive control wells and BRET ratioₘₐₓ is the BRET ratio from the DMSO treated negative control well.

The % inhibition values were plotted as a function of compound concentration and the following 4-parameter fit was applied to derive the IC₅₀ values:

$$Y = Bottom + \frac{( Top - Bottom )}{( 1 + {( \frac{X}{{IC}_{50}} )}^{Hill Coefficient}}$$

where top and bottom are normally allowed to float, but may be fixed at 100 or 0 respectively in a 3-parameter fit. The Hill Coefficient is normally allowed to float but may also be fixed at 1 in a 3-parameter fit. Y is the % inhibition and X is the compound concentration.

### Validation of NanoBRET Probe Displacement Assays

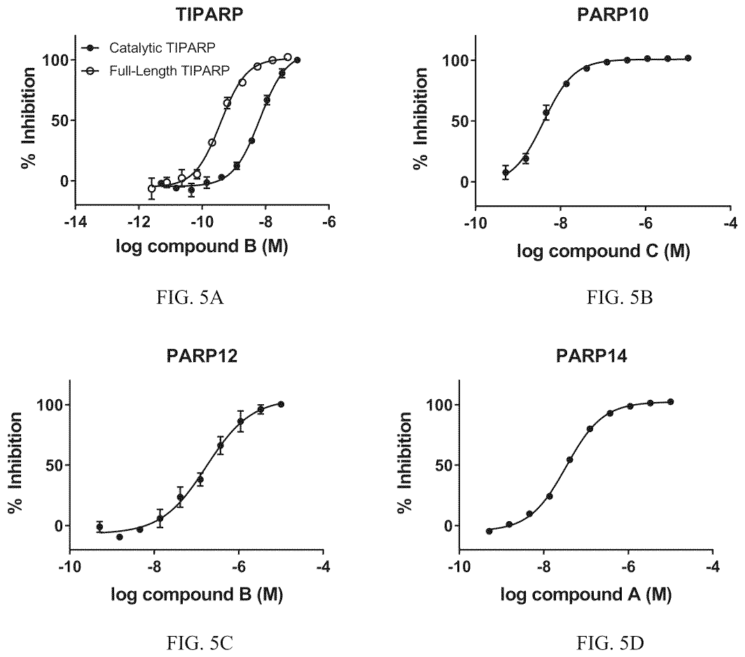

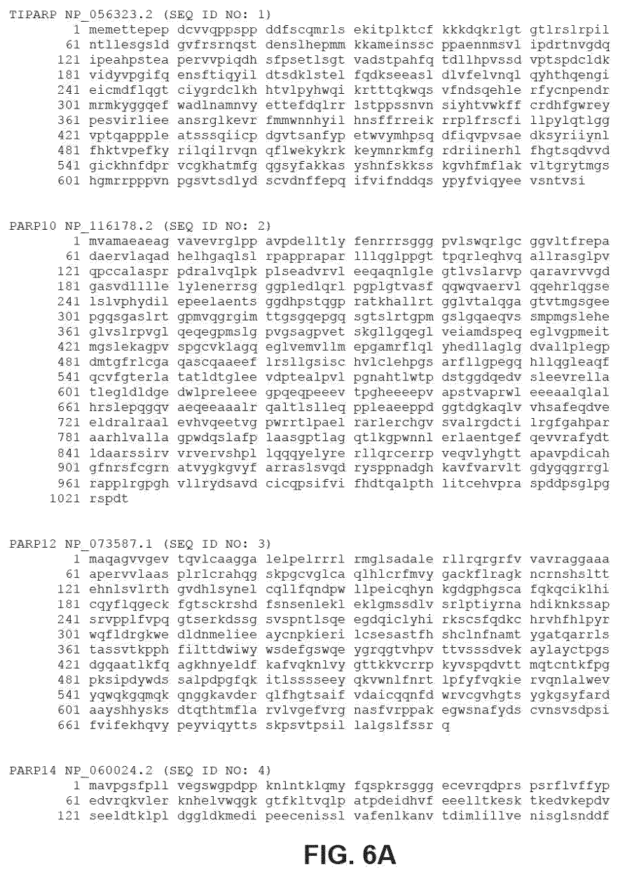

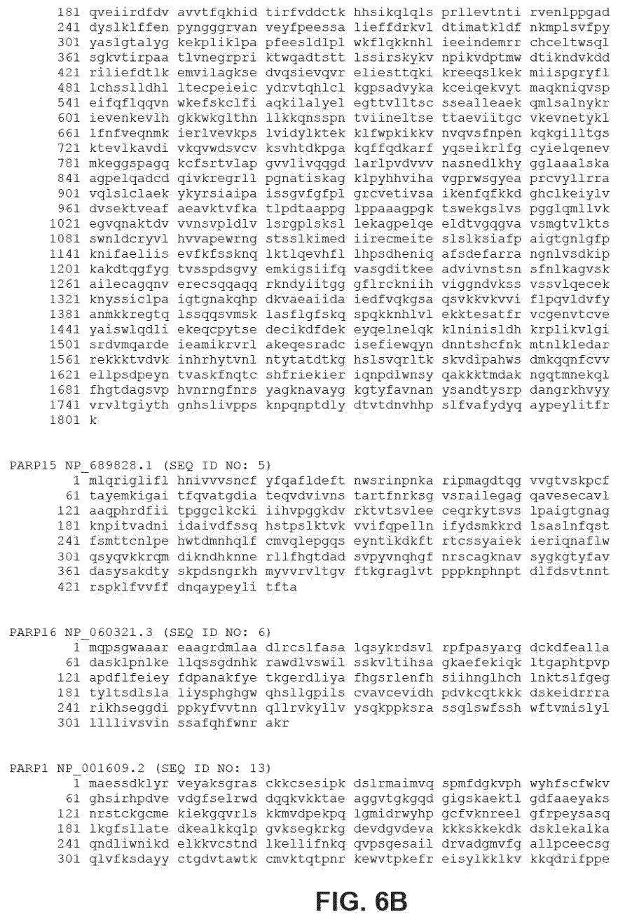

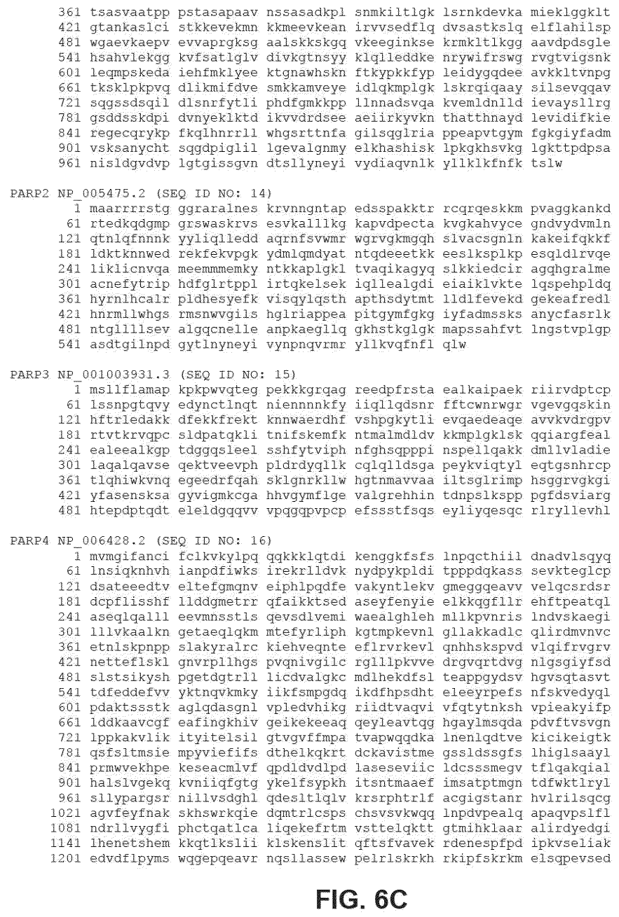

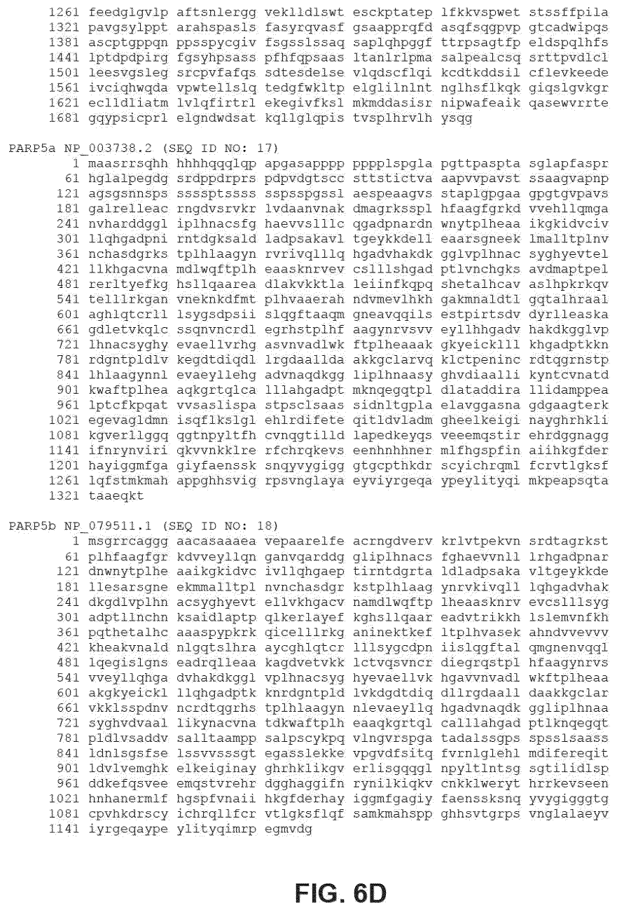

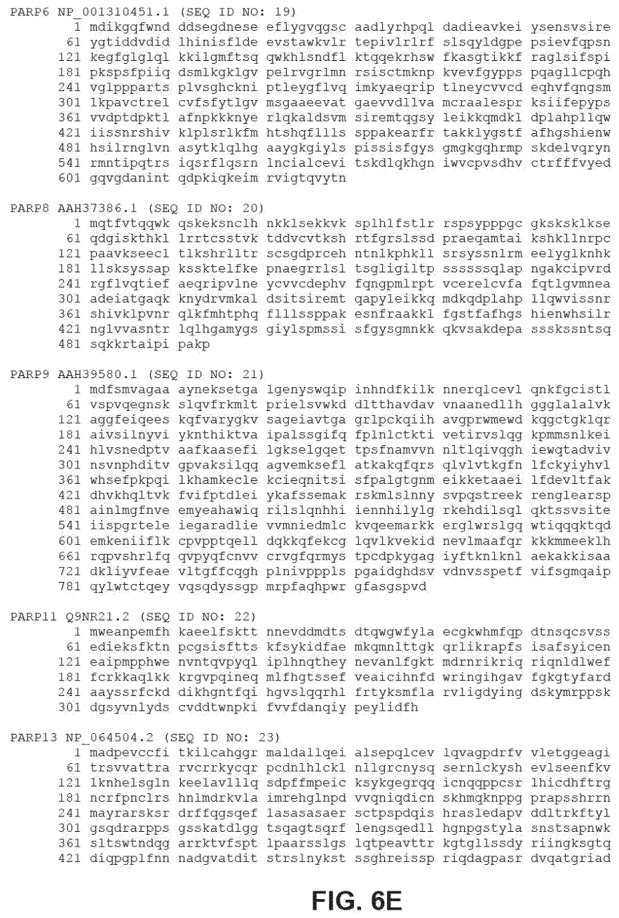

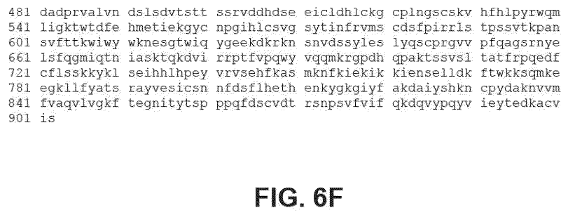

The probe displacement assays were validated by outcompeting Probe A with Compound A in the PARP14 NanoBRET assay, Compound B in the TIPARP NanoBRET assay, Compound C in the PARP10 NanoBRET assay and Compound B in the PARP12 NanoBRET assay. The compounds used to test the probe displacement of Probe A are analogs that do not contain a linker or fluorescent tag. The assays for TIPARP full-length and catalytic domain, PARP10, PARP12 and PARP14 were set up as described above, and the results are shown in FIG. 5.

FIGS. 5A-5D are validation results of the NanoBRET probe displacement binding assays. Dose response curves for control compounds were generated using each assay to confirm that Probe A was able to be outcompeted from the monoPARP enzyme. IC₅₀ values were full-length TIPARP Compound B=4 nM, catalytic domain TIPARP Compound B=7 nM, PARP10 Compound C=4 nM, PARP12 Compound B=170 nM, PARP14 compound A=30 nM.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

### REFERENCES

1. Leidecker, O.; Bonfiglio, J. J.; Colby, T.; Zhang, Q.; Atanassov, I.; Zaja, R.; Palazzo, L.; Stockum, A.; Ahel, I.; Matic, I., Serine is a new target residue for endogenous ADP-ribosylation on histones. Nat Chem Biol 2016, 12 (12), 998-1000.
2. Belousova, E. A.; Ishchenko capital A, C.; Lavrik, O. I., Dna is a New Target of Parp3. Sci Rep 2018, 8 (1), 4176.
3. Yamada, T.; Horimoto, H.; Kameyama, T.; Hayakawa, S.; Yamato, H.; Dazai, M.; Takada, A.; Kida, H.; Bott, D.; Zhou, A. C.; Hutin, D.; Watts, T. H.; Asaka, M.; Matthews, J.; Takaoka, A., Constitutive aryl hydrocarbon receptor signaling constrains type I interferon-mediated antiviral innate defense. Nat Immunol 2016, 17 (6), 687-94.
4. Caprara, G.; Prosperini, E.; Piccolo, V.; Sigismondo, G.; Melacarne, A.; Cuomo, A.; Boothby, M.; Rescigno, M.; Bonaldi, T.; Natoli, G., PARP14 Controls the Nuclear Accumulation of a Subset of Type I IFN-Inducible Proteins. J Immunol 2018, 200 (7), 2439-2454.
5. Zaffini, R.; Gotte, G.; Menegazzi, M., Asthma and poly(ADP-ribose) polymerase inhibition: a new therapeutic approach. Drug Des Devel Ther 2018, 12, 281-293.
6. Jwa, M.; Chang, P., PARP16 is a tail-anchored endoplasmic reticulum protein required for the PERK- and IRE1alpha-mediated unfolded protein response. Nat Cell Biol 2012, 14 (11), 1223-30.
7. Barbarulo, A.; Iansante, V.; Chaidos, A.; Naresh, K.; Rahemtulla, A.; Franzoso, G.; Karadimitris, A.; Haskard, D. O.; Papa, S.; Bubici, C., Poly(ADP-ribose) polymerase family member 14 (PARP14) is a novel effector of the JNK2-dependent pro-survival signal in multiple myeloma. Oncogene 2013, 32 (36), 4231-42.
8 Thorsell, A. G.; Ekblad, T.; Karlberg, T.; Low, M.; Pinto, A. F.; Tresaugues, L.; Moche, M.; Cohen, M. S.; Schuler, H., Structural Basis for Potency and Promiscuity in Poly(ADP-ribose) Polymerase (PARP) and Tankyrase Inhibitors. J Med Chem 2017, 60 (4), 1262-1271.
9. Copeland, R. A., Evaluation of enzyme inhibitors in drug discovery. A guide for medicinal chemists and pharmacologists. Methods Biochem Anal 2005, 46, 1-265.
10. Wahlberg, E.; Karlberg, T.; Kouznetsova, E.; Markova, N.; Macchiarulo, A.; Thorsell, A. G.; Pol, E.; Frostell, A.; Ekblad, T.; Oncu, D.; Kull, B.; Robertson, G. M.; Pellicciari, R.; Schuler, H.; Weigelt, J., Family-wide chemical profiling and structural analysis of PARP and tankyrase inhibitors. Nat Biotechnol 2012, 30 (3), 283-8.
11. Davis, B. J.; Erlanson, D. A., Learning from our mistakes: the ‘unknown knowns’ in fragment screening. Bioorg Med Chem Lett 2013, 23 (10), 2844-52.

1-14. (canceled)

15. A method of identifying an inhibitor for Poly (ADP-ribose) polymerase (PARP), the method comprising:
contacting a polypeptide comprising a PARP catalytic domain with a PARP probe in the presence of a test compound, wherein the PARP probe comprises a fluorophore;
exposing the probe to polarized excitation light, thereby generating fluorescence;
determining a fluorescence polarization value of the fluorescence; and
identifying the test compound as an inhibitor for PARP based on the fluorescence polarization value of the fluorescence.

16. The method of claim 15, further comprising identifying the test compound as an inhibitor for PARP if the fluorescence polarization value of the fluorescence is decreased as compared to a reference level.

17. The method of claim 16, wherein the reference value is the fluorescence polarization value of the fluorescence in the absence of the test compound.

18. The method of claim 15, wherein the PARP probe is a compound having the structure: 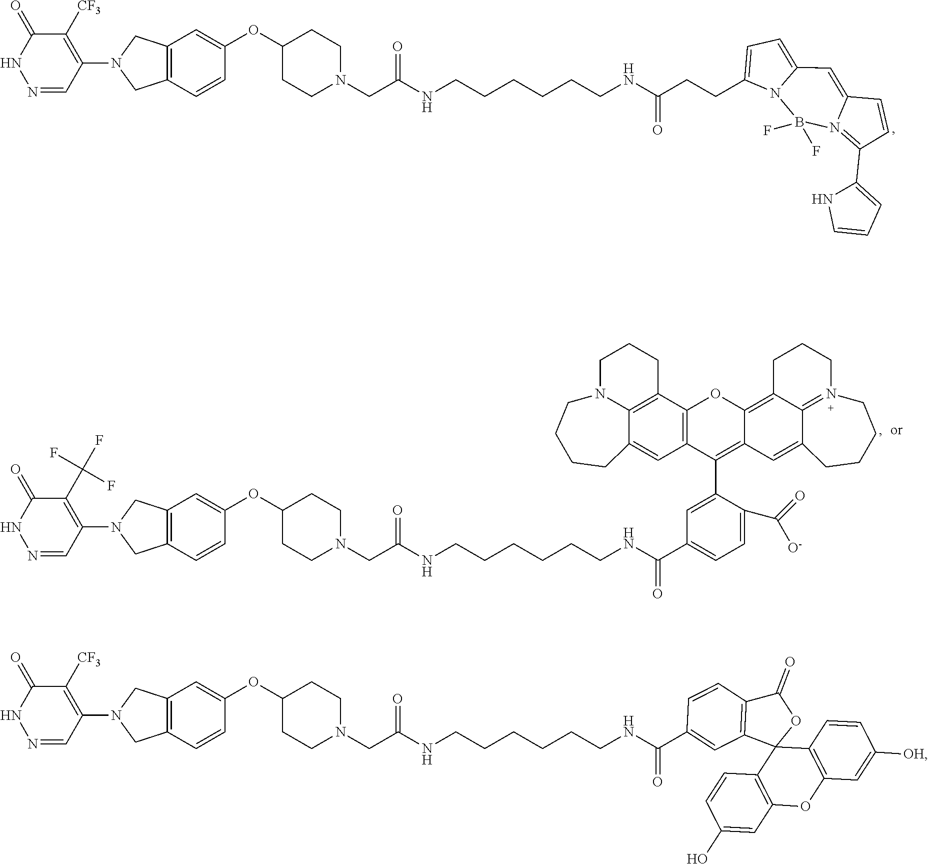
or a salt thereof.

19. The method of claim 15, wherein the PARP is PARP1, PARP2, PARP3, PARP4, PARP5a, PARP5b, PARP6, TIPARP, PARP8, PARP10, PARP11, PARP12, PARP13, PARP14, PARP15, or PARP16.

20. The method of claim 15, wherein the test compound is a small molecule, a nucleic acid, a polypeptide, or an antibody or antigen-binding fragment thereof.

21-37. (canceled)
